(19) United States
(12) Patent Application Publication
(10) Pub. No.: US 2004/0097482 A1
(43) Pub. Date: May 20, 2004

(54) Combinations of nicotinic acid and derivatives thereof and sterol absorption inhibitor(s) and treatments for vascular indications

(75) Inventors: Harry R. Davis, Berkeley Heights, NJ (US); Teddy Kosoglou, Jamison, PA (US); Michael P. Graziano, Scotch Plains, NJ (US); Joseph A. Hedrick, South River, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: Schering Corporation

(21) Appl. No.: 10/639,900

(22) Filed: Aug. 13, 2003

Related U.S. Application Data
(63) Continuation of application No. 10/057,646, filed on Jan. 25, 2002.
(60) Provisional application No. 60/264,275, filed on Jan. 26, 2001.
(60) Provisional application No. 60/323,842, filed on Sep. 21, 2001.

(51) Int. Cl. A61K 31/397

(52) U.S. Cl. 514/210.02

(57) ABSTRACT

The present invention provides compositions, therapeutic combinations and methods including: (a) at least one HM74 or HM74A agonist and (b) at least one substituted azetidinone or substituted β-lactam sterol absorption inhibitor which can be useful for treating vascular conditions, diabetes, obesity and lowering plasma levels of sterols.

### CROSS-REFERENCE TO RELATED APPLICATIONS

This application is a continuation-in-part of U.S. patent application Ser. No. 10/057,646; filed Jan. 25, 2002 which claims the benefit of priority from U.S. Provisional Patent Application Serial No. 60/264,275 filed Jan. 26, 2001 and U.S. Provisional Patent Application Serial No. 60/323,842 filed Sep. 21, 2001, each of which is incorporated herein by reference in its entirety.

### FIELD OF THE INVENTION

The present invention relates to compositions and therapeutic combinations comprising agonists of the HM74A receptor, such as nicotinic acid or derivatives thereof, and certain sterol absorption inhibitors for treating hyperlipidemic conditions such as are associated with atherosclerosis, hypercholesterolemia and other vascular conditions in subjects.

### BACKGROUND OF THE INVENTION

Atherosclerotic coronary heart disease (CHD) represents the major cause for death and vascular morbidity in the western world. Risk factors for atherosclerotic coronary heart disease include hypertension, diabetes mellitus, family history, male gender, cigarette smoke and serum cholesterol. A total cholesterol level in excess of 225-250 mg/dl is associated with significant elevation of risk of CHD.

Cholesteryl esters are a major component of atherosclerotic lesions and the major storage form of cholesterol in arterial wall cells. Formation of cholesteryl esters is also a key step in the intestinal absorption of dietary cholesterol. Thus, inhibition of cholesteryl ester formation and reduction of serum cholesterol can inhibit the progression of atherosclerotic lesion formation, decrease the accumulation of cholesteryl esters in the arterial wall, and block the intestinal absorption of dietary cholesterol.

The regulation of whole-body cholesterol homeostasis in mammals and animals involves the regulation of dietary cholesterol and modulation of cholesterol biosynthesis, bile acid biosynthesis and the catabolism of the cholesterol-containing plasma lipoproteins. The liver is the major organ responsible for cholesterol biosynthesis and catabolism and, for this reason, it is a prime determinant of plasma cholesterol levels. The liver is the site of synthesis and secretion of very low density lipoproteins (VLDL) which are subsequently metabolized to low density lipoproteins (LDL) in the circulation. LDL are the predominant cholesterol-carrying lipoproteins in the plasma and an increase in their concentration is correlated with increased atherosclerosis. When intestinal cholesterol absorption is reduced, by whatever means, less cholesterol is delivered to the liver. The consequence of this action is decreased hepatic lipoprotein (VLDL) production and an increase in the hepatic clearance of plasma cholesterol, mostly as LDL. Thus, the net effect of inhibiting intestinal cholesterol absorption is a decrease in plasma cholesterol levels.

Nicotinic acid and its derivatives can inhibit hepatic production of VLDL and its metabolite LDL and increases HDL and apo A-1 levels.

U.S. Pat. Nos. 5,767,115, 5,624,920, 5,668,990, 5,656,624 and 5,688,787, respectively, disclose hydroxy-substituted azetidinone compounds and substituted β-lactam compounds useful for lowering cholesterol and/or in inhibiting the formation of cholesterol-containing lesions in mammalian arterial walls. U.S. Pat. Nos. 5,846,966 and 5,661,145, respectively, disclose hydroxy-substituted azetidinone compounds or substituted β-lactam compounds in combination with HMG CoA reductase inhibitors for preventing or treating atherosclerosis and reducing plasma cholesterol levels.

PCT Patent Application No. WO 00/38725 discloses cardiovascular therapeutic combinations including an ileal bile acid transport inhibitor or cholesteryl ester transport protein inhibitor in combination with a fibric acid derivative, nicotinic acid derivative, microsomal triglyceride transfer protein inhibitor, cholesterol absorption antagonist, phytosterol, stanol, antihypertensive agent or bile acid sequestrant.

U.S. Pat. No. 5,698,527 discloses ergostanone derivatives substituted with disaccharides as cholesterol absorption inhibitors, employed alone or in combination with certain other cholesterol lowering agents, which are useful in the treatment of hypercholesterolemia and related disorders.

Despite recent improvements in the treatment of vascular disease, there remains a need in the art for improved compositions and treatments for hyperlipidaemia, atherosclerosis and other vascular conditions.

### SUMMARY OF THE INVENTION

One embodiment of the present invention provides a composition comprising:

(a) at least one HM74 or HM74A agonist; and

(b) at least one sterol absorption inhibitor selected from the group consisting of:

(i) a compound represented by Formula (I):

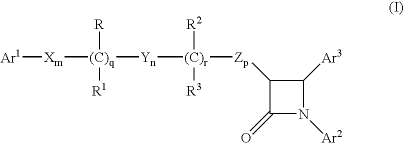

or pharmaceutically acceptable salts or solvates thereof,

wherein in Formula (I) above:

Ar¹ and Ar² are independently selected from the group consisting of aryl and R⁴-substituted aryl;

Ar³ is aryl or R⁵-substituted aryl;

X, Y and Z are independently selected from the group consisting of —CH₂—, —CH(lower alkyl)- and —C(dilower alkyl)-;

R and R² are independently selected from the group consisting of —OR⁶, —O(CO)R⁶, —O(CO)OR⁹ and —O(CO)NR⁶R⁷;

R¹ and R³ are independently selected from the group consisting of hydrogen, lower alkyl and aryl;

q is 0 or 1;

r is 0 or 1;

m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;

R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶, —CH═CH—COOR⁶, —CF₃, —CN, —NO₂ and halogen;

R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;

R⁶, R⁷ and R⁸ are independently selected from the group consisting of hydrogen, lower alkyl, aryl and aryl-substituted lower alkyl; and

R⁹ is lower alkyl, aryl or aryl-substituted lower alkyl;

(ii) a compound represented by Formula (III):

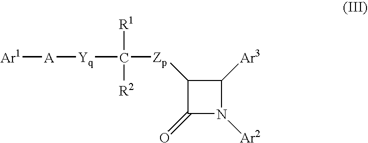

or pharmaceutically acceptable salts or solvates thereof wherein, in Formula (III) above:

Ar¹ is R³-substituted aryl;

Ar² is R⁴-substituted aryl;

Ar³ is R⁵-substituted aryl;

Y and Z are independently selected from the group consisting of —CH₂—, —CH(lower alkyl)- and —C(dilower alkyl)-;

A is selected from —O—, —S—, —S(O)— or —S(O)₂—;

R¹ is selected from the group consisting of —OR⁶, —O(CO)R⁶, —O(CO)OR⁹ and —O(CO)NR⁶R⁷; R² is selected from the group consisting of hydrogen, lower alkyl and aryl; or R¹ and R² together are ═O;

q is 1, 2 or 3;

p is 0, 1, 2, 3 or 4;

R⁵ is 1-3 substituents independently selected from the group consisting of —OR⁶, O(CO)R⁶, O(CO)OR⁹, —O(CH₂)₁₋₅OR⁹, —O(CO)NR⁶R⁷, —NR⁶R⁷, —NR⁶(CO)R⁷, —NR⁶(CO)OR⁹, —NR⁶(CO)NR⁷R⁸, —NR⁶SO₂-lower alkyl, —NR⁶SO₂-aryl, —CONR⁶R⁷, —COR⁶, —SO₂NR⁶R⁷, S(O)₀₋₂-alkyl, S(O)₀₋₂-aryl, —O(CH₂)₁₋₁₀—COOR⁶, —O(CH₂)₁₋₁₀CNR⁶R⁷, o-halogeno, m-halogeno, o-lower alkyl, m-lower alkyl, -(lower alkylene)-COOR⁶, and —CH═CH—COOR⁶;

R³ and R⁴ are independently 1-3 substituents independently selected from the group consisting of R⁵, hydrogen, p-lower alkyl, aryl, —NO₂, —CF₃ and p-halogeno;

R⁶, R⁷ and R⁸ are independently selected from the group consisting of hydrogen, lower alkyl, aryl and aryl-substituted lower alkyl; and

R⁹ is lower alkyl, aryl or aryl-substituted lower alkyl;

(iii) a compound represented by Formula (IV):

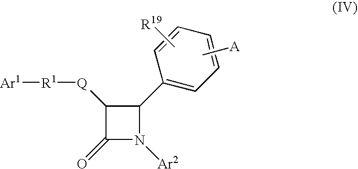

or pharmaceutically acceptable salts or solvates wherein, in Formula (IV) above:

A is selected from the group consisting of R²-substituted heterocycloalkyl, R²-substituted heteroaryl, R²-substituted benzofused heterocycloalkyl, and R²-substituted benzofused heteroaryl;

Ar¹ is aryl or R³-substituted aryl;

Ar² is aryl or R⁴-substituted aryl;

Q is a bond or, with the 3-position ring carbon of the azetidinone, forms the spiro group

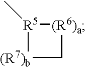

and

R¹ is selected from the group consisting of:

—(CH₂)_{q}—, wherein q is 2-6, provided that when Q forms a spiro ring, q can also be zero or 1;

—(CH₂)ₑ-G-(CH₂)ᵣ—, wherein G is —O—, —C(O)—, phenylene, —NR⁸— or —S(O)₀₋₂—, e is 0-5 and r is 0-5, provided that the sum of e and r is 1-6;

—(C₂-C₆ alkenylene)-; and

'(CH₂)_{f}-V-(CH₂)_{g}—, wherein V is C₃-C₆ cycloalkylene, f is 1-5 and g is 0-5, provided that the sum of f and g is 1-6;

R⁵ is selected from:

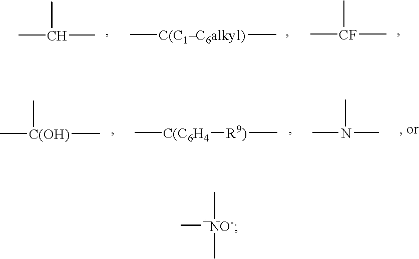

R⁶ and R⁷ are independently selected from the group consisting of —CH₂—, —CH(C₁-C₆ alkyl)-, —C(di-(C₁-C₆) alkyl), —CH═CH— and —C(C₁-C₆ alkyl)═CH—; or R⁵ together with an adjacent R⁶, or R⁵ together with an adjacent R⁷, form a —CH═CH— or a —CH═C(C₁-C₆ alkyl)- group;

a and b are independently 0, 1, 2 or 3, provided both are not zero; provided that when R⁶ is —CH═CH— or —C(C₁-C₆ alkyl)═CH—, a is 1; provided that when R⁷ is —CH═CH— or —C(C₁-C₆ alkyl)═CH—, b is 1; provided that when a is 2 or 3, the R⁶'s can be the same or different; and provided that when b is 2 or 3, the R⁷'s can be the same or different;

and when Q is a bond, R¹ also can be selected from:

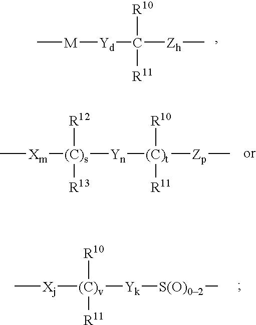

where M is —O—, —S—, —S(O)— or —S(O)₂—;

X, Y and Z are independently selected from the group consisting of —CH₂—, —CH(C₁-C₆ alkyl)- and —C(di-(C₁-C₆) alkyl);

R¹⁰ and R¹² are independently selected from the group consisting of —OR¹⁴, —O(CO)R¹⁴, —O(CO)OR¹⁶ and —O(CO)NR¹⁴R¹⁵;

R¹¹ and R¹³ are independently selected from the group consisting of hydrogen, (C₁-C₆)alkyl and aryl; or R¹⁰ and R¹¹ together are ═O, or R¹² and R¹³ together are ═O;

d is 1, 2 or 3;

h is 0, 1, 2, 3 or 4;

s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-, —NHC(O)R¹⁶, OH, C₁-C₆ alkoxy, —OC(O)R¹⁶, —COR¹⁴, hydroxy(C₁-C₆)alkyl, (C₁-C₆)alkoxy(C₁-C₆)alkyl, NO₂, —S(O)₀₋₂R^{≠}, —SO₂NR¹⁴R¹⁵ and —(C₁-C₆ alkylene)COOR¹⁴; when R² is a substituent on a heterocycloalkyl ring, R² is as defined, or is ═O or

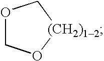

and, where R² is a substituent on a substitutable ring nitrogen, it is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸,

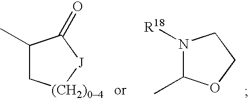

wherein J is —O—, —NH—, —NR¹⁸— or —CH₂—;

R³ and R⁴ are independently selected from the group consisting of 1-3 substituents independently selected from the group consisting of (C₁-C₆)alkyl, —OR¹⁴, —O(CO)R¹⁴, —O(CO)OR¹⁶, —O(CH₂)₁₋₅OR¹⁴, —O(CO)NR¹⁴R¹⁵, —NR¹⁴R¹⁵, —NR¹⁴(CO)R¹⁵, —NR¹⁴(CO)OR¹⁶, —NR¹⁴(CO)NR¹⁵R¹⁹, —NR¹⁴SO₂R¹⁶, —COOR¹⁴, —CONR¹⁴R¹⁵, —COR¹⁴, —SO₂NR¹⁴R¹⁵, S(O)₀₋₂R¹⁶, —O(CH₂)₁₋₁₀—COOR¹⁴, —O(CH₂)₁₋₁₀CONR¹⁴R¹⁵, —(C₁-C₆ alkylene)-COOR¹⁴, —CH═CH—COOR¹⁴, —CF₃, —CN, —NO₂ and halogen;

R⁸ is hydrogen, (C₁-C₆)alkyl, aryl (C₁-C₆)alkyl, —C(O)R¹⁴ or —COOR¹⁴;

R⁹ and R¹⁷ are independently 1-3 groups independently selected from the group consisting of hydrogen, (C₁-C₆)alkyl, (C₁-C₆)alkoxy, —COOH, NO₂, —NR¹⁴R¹⁵, OH and halogeno;

R¹⁴ and R¹⁵ are independently selected from the group consisting of hydrogen, (C₁-C₆)alkyl, aryl and aryl-substituted (C₁-C₆)alkyl;

R¹⁶ is (C₁-C₆)alkyl, aryl or R¹⁷-substituted aryl;

R¹⁸ is hydrogen or (C₁-C₆)alkyl; and

R¹⁹ is hydrogen, hydroxy or (C₁-C₆)alkoxy;

(iv) a compound represented by Formula (V):

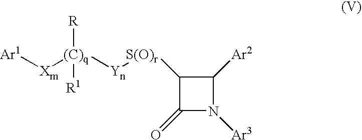

or pharmaceutically acceptable salts or solvates thereof, wherein, in Formula (V) above:

Ar¹ is aryl, R¹⁰-substituted aryl or heteroaryl;

Ar² is aryl or R⁴-substituted aryl;

Ar³ is aryl or R⁵-substituted aryl;

X and Y are independently selected from the group consisting of —CH₂—, —CH(lower alkyl)- and —C(dilower alkyl)-;

R is —OR⁶, —O(CO)R⁶, —O(CO)OR⁹ or —O(CO)NR⁶R⁷; R¹ is hydrogen, lower alkyl or aryl; or R and R¹ together are ═O;

q is 0 or 1;

r is 0, 1 or 2;

m and n are independently 0, 1, 2, 3, 4 or 5; provided that the sum of m, n and q is 1, 2, 3, 4 or 5;

R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;

R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂, halogen, -(lower alkylene)COOR⁶ and —CH═CH—COOR⁶;

R⁶, R⁷ and R⁸ are independently selected from the group consisting of hydrogen, lower alkyl, aryl and aryl-substituted lower alkyl;

R⁹ is lower alkyl, aryl or aryl-substituted lower alkyl; and

R¹⁰ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR(CO)NR⁷R⁸, —NR⁶SO₂R⁹, —COOR⁶, —CONR⁶R⁷, —COR⁶, —SO₂NR⁶R⁷, —S(O)₀₋₂R⁹, —O(CH₂)₁₋₁₀—COOR⁶, —O(CH₂)₁₋₁₀CONR⁶R⁷, —CF₃, —CN, —NO₂ and halogen;

(v) a compound represented by Formula (VI):

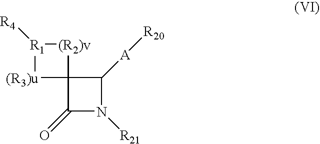

or pharmaceutically acceptable salts or solvates thereof, wherein:

R₁ is

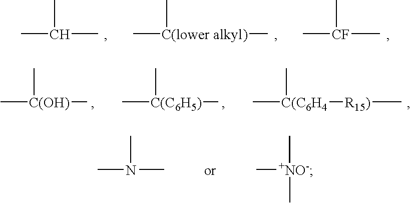

R₂ and R₃ are independently selected from the group consisting of: —CH₂—, —CH(lower alkyl)-, —C(di-lower alkyl)-, —CH═CH— and —C(lower alkyl)═CH—; or R₁ together with an adjacent R₂, or R₁ together with an adjacent R₃, form a —CH═CH— or a —CH═C(lower alkyl)- group;

u and v are independently 0, 1, 2 or 3, provided both are not zero; provided that when R₂ is —CH═CH— or —C(lower alkyl)═CH—, v is 1; provided that when R₃ is —CH═CH— or —C(lower alkyl)═CH—, u is 1; provided that when v is 2 or 3, the R₂'s can be the same or different; and provided that when u is 2 or 3, the R₃'s can be the same or different;

R₄ is selected from B—(CH₂)ₘC(O)—, wherein m is 0, 1, 2, 3, 4 or 5;

B—(CH₂)_{q}—, wherein q is 0, 1, 2, 3, 4, 5 or 6;

B—(CH₂)ₑ-Z-(CH₂)ᵣ—, wherein Z is —O—, —C(O)—, phenylene, —N(R₈)— or —S(O)₀₋₂-, e is 0, 1, 2, 3, 4 or 5 and r is 0, 1, 2, 3, 4 or 5, provided that the sum of e and r is 0, 1, 2, 3, 4, 5 or 6;

B—(C₂-C₆ alkenylene)-;

B—(C₄-C₆ alkadienylene)-;

B—(CH₂)ₜ-Z-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6;

B—(CH₂)_{f}-V-(CH₂)_{g}—, wherein V is C₃-C₆ cycloalkylene, f is 1, 2, 3, 4 or 5 and g is 0, 1, 2, 3, 4 or 5, provided that the sum of f and g is 1, 2, 3, 4, 5 or 6;

B—(CH₂)ₜ-V-(C₂-C₆ alkenylene)- or

B—(C₂-C₆ alkenylene)-V-(CH₂)ₜ—, wherein V and t are as defined above, provided that the sum of t and the number of carbon atoms in the alkenylene chain is 2, 3, 4, 5 or 6;

B—(CH₂)ₐ-Z-(CH₂)_{b}-V-(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or

T-(CH₂)ₛ—, wherein T is cycloalkyl of 3-6 carbon atoms and s is 0, 1, 2, 3, 4, 5 or 6; or

R₁ and R₄ together form the group

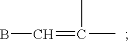

B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or

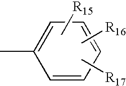

W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R₇-benzyl, benzyloxy, R₇-benzyloxy, phenoxy, R₇-phenoxy, dioxolanyl, NO₂, —N(R₈)(R₉), N(R₈)(R₉)-lower alkylene-, N(R₈)(R₉)-lower alkylenyloxy-, OH, halogeno, —CN, —N₃, —NHC(O)OR₁₀, —NHC(O)R₁₀, R₁₁O₂SNH—, (R₁₁O₂S)₂N—, —S(O)₂NH₂, —S(O)₀₋₂R₈, tert-butyldimethyl-silyloxymethyl, —C(O)R₁₂, —COOR₁₉, —CON(R₈)(R₉), —CH═CHC(O)R₁₂, -lower alkylene-C(O)R₁₂, R₁₀C(O)(lower alkylenyloxy)-, N(R₈)(R₉)C(O)(lower alkylenyloxy)- and

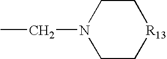

for substitution on ring carbon atoms, and the substituents on the substituted heteroaryl ring nitrogen atoms, when present, are selected from the group consisting of lower alkyl, lower alkoxy, C(O)OR₁₀, —C(O)R₁₀, OH, N(R₈)(R₉)-lower alkylene-, N(R₈)(R₉)-lower alkylenyloxy-, —S(O)₂NH₂ and 2-(trimethylsilyl)-ethoxymethyl;

R₇ is 1-3 groups independently selected from the group consisting of lower alkyl, lower alkoxy, —COOH, NO₂, —N(R₈)(R₉), OH, and halogeno;

R₈ and R₉ are independently selected from H or lower alkyl;

R₁₀ is selected from lower alkyl, phenyl, R₇-phenyl, benzyl or R₇-benzyl;

R₁₁ is selected from OH, lower alkyl, phenyl, benzyl, R₇-phenyl or R₇-benzyl;

R₁₂ is selected from H, OH, alkoxy, phenoxy, benzyloxy,

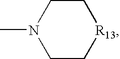

—N(R₈)(R₉), lower alkyl, phenyl or R₇-phenyl;

R₁₃ is selected from —O—, —CH₂—, —NH—, —N(lower alkyl)- or —NC(O)R₁₉;

R₁₅, R₁₆ and R₁₇ are independently selected from the group consisting of H and the groups defined for W; or R₁₅ is hydrogen and R₁₆ and R₁₇, together with adjacent carbon atoms to which they are attached, form a dioxolanyl ring;

R₁₉ is H, lower alkyl, phenyl or phenyl lower alkyl; and

R₂₀ and R₂₁ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzofused heteroaryl, W-substituted benzofused heteroaryl and cyclopropyl, wherein heteroaryl is as defined above;

(vi) a compound represented by Formula (VIIA) or (VIIB):

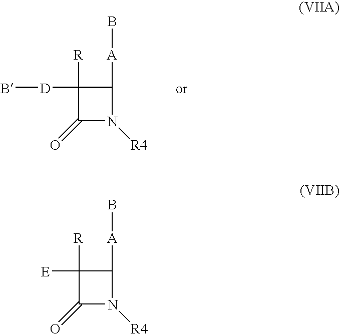

or pharmaceutically acceptable salts or solvates thereof, wherein in Formulae (VIIA) and (VIIB):

A is —CH═CH—, —C≡C— or —(CH₂)ₚ— wherein p is 0, 1 or 2;

B is

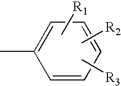

B′ is

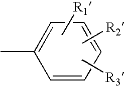

D is —(CH₂)ₘC(O)— or —(CH₂)_{q}— wherein m is 1, 2, 3 or 4 and q is 2, 3 or 4;

E is C₁₀ to C₂₀ alkyl or —C(O)—(C₉ to C₁₉)-alkyl, wherein the alkyl is straight or branched, saturated or containing one or more double bonds;

R is hydrogen, C₁-C₁₅ alkyl, straight or branched, saturated or containing one or more double bonds, or B—(CH₂)ᵣ—, wherein r is 0, 1, 2, or 3;

R₁, R₂, R₃, R_{1′}, R_{2′}, and R_{3′} are independently selected from the group consisting of hydrogen, lower alkyl, lower alkoxy, carboxy, NO₂, NH₂, OH, halogeno, lower alkylamino, dilower alkylamino, —NHC(O)OR₅, R₆O₂SNH— and —S(O)₂NH₂;

R₄ is

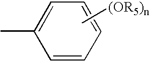

wherein n is 0, 1, 2 or 3;

R₅ is lower alkyl; and

R₆ is OH, lower alkyl, phenyl, benzyl or substituted phenyl wherein the substituents are 1-3 groups independently selected from the group consisting of lower alkyl, lower alkoxy, carboxy, NO₂, NH₂, OH, halogeno, lower alkylamino and dilower alkylamino;

(vii) a compound represented by Formula (VIII):

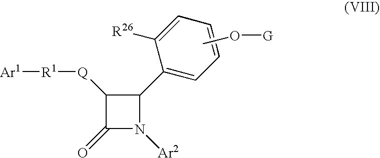

or pharmaceutically acceptable salts or solvates thereof, wherein, in Formula (VIII) above,

R²⁶ is H or OG¹;

G and G¹ are independently selected from the group consisting of

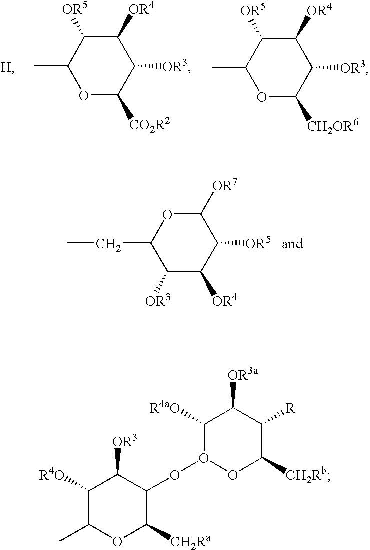

provided that when R²⁶ is H or OH, G is not H;

R, R^{a} and R^{b} are independently selected from the group consisting of H, —OH, halogeno, —NH₂, azido, (C₁-C₆)alkoxy(C₁-C₆)-alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are independently selected from the group consisting of H, (C₁-C₆)alkyl, aryl and aryl(C₁-C₆)alkyl;

R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;

R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C7)cycloalkyl and R³²-substituted-(C₃-C7)cycloalkyl(C₁-C₆)alkyl;

R³¹ is selected from the group consisting of H and (C₁-C₄)alkyl;

T is selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;

R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

Ar¹ is aryl or R¹⁰-substituted aryl;

Ar² is aryl or R¹¹-substituted aryl;

Q is a bond or, with the 3-position ring carbon of the azetidinone, forms the spiro group

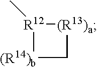

and

R¹ is selected from the group consisting of

—(CH₂)_{q}—, wherein q is 2-6, provided that when Q forms a spiro ring, q can also be zero or 1;

—(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;

—(C₂-C₆)alkenylene-; and

—(CH₂)_{f}-V-(CH₂)_{g}—, wherein V is C₃-C₆ cycloalkylene, f is 1-5 and g is 0-5, provided that the sum of f and g is 1-6;

R¹² is

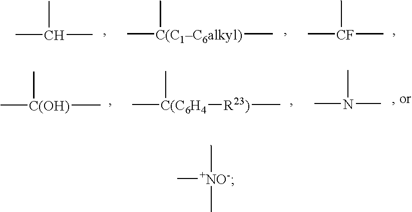

R¹³ and R¹⁴ are independently selected from the group consisting of —CH₂—, —CH(C₁-C₆ alkyl)-, —C(di-(C₁-C₆)alkyl), —CH═CH— and —C(C₁-C₆ alkyl)═CH—; or R¹² together with an adjacent R¹³, or R¹² together with an adjacent R¹⁴, form a —CH═CH— or a —CH═C(C₁-C₆ alkyl)- group;

a and b are independently 0, 1, 2 or 3, provided both are not zero;

provided that when R¹³ is —CH═CH— or —C(C₁-C₆ alkyl)═CH—, a is 1;

provided that when R¹⁴ is —CH═CH— or —C(C₁-C₆ alkyl)═CH—, b is 1;

provided that when a is 2 or 3, the R¹³'s can be the same or different; and

provided that when b is 2 or 3, the R¹⁴'s can be the same or different;

and when Q is a bond, R¹ also can be:

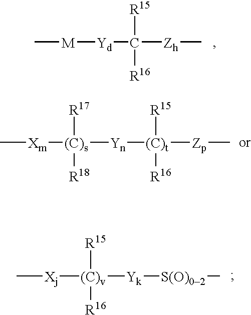

M is —O—, —S—, —S(O)— or —S(O)₂—;

X, Y and Z are independently selected from the group consisting of —CH₂—, —CH(C₁-C₆)alkyl- and —C(di-(C₁-C₆)alkyl);

R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀-COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;

R¹⁵ and R¹⁷ are independently selected from the group consisting of —OR¹⁹, —O(CO)R¹⁹, —O(CO)OR²¹ and —O(CO)NR¹⁹R²⁰;

R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;

d is 1, 2 or 3;

h is 0, 1, 2, 3 or 4;

s is 0 or 1; t is 0 or 1; m, n and p are independently 0-4;

provided that at least one of s and t is 1, and the sum of m, n, p, s and t is 1-6;

provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

and when Q is a bond and R¹ is

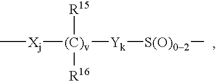

Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —COOR¹⁹;

R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and

R²⁵ is H, —OH or (C₁-C₆)alkoxy; and

(viii) a compound represented by Formula (IX):

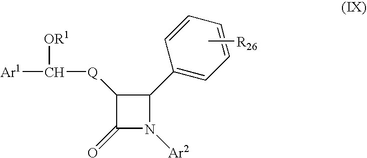

or pharmaceutically acceptable salts or solvates thereof, wherein in Formula (IX):

R²⁶ is selected from the group consisting of:

a) OH;

b) OCH₃;

c) fluorine and

d) chlorine.

R¹ is selected from the group consisting of

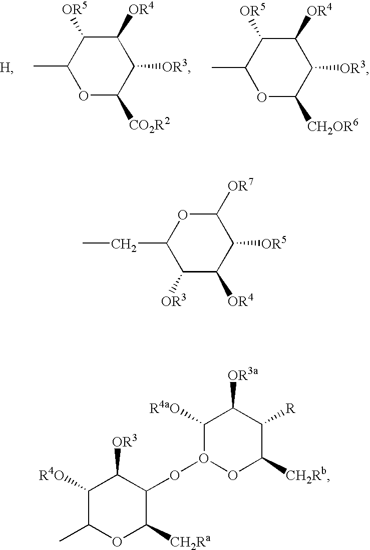

—SO₃H; natural and unnatural amino acids.

R, R^{a} and R^{b} are independently selected from the group consisting of H, —OH, halogeno, —NH₂, azido, (C₁-C₆)alkoxy(C₁-C₆)-alkoxy and —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are independently selected from the group consisting of H, (C₁-C₆)alkyl, aryl and aryl(C₁-C₆)alkyl;

R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;

R³⁰ is independently selected form the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is independently selected from the group consisting of H and (C₁-C₄)alkyl;

T is independently selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;

R³² is independently selected from 1-3 substituents independently selected from the group consisting of H, halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

Ar¹ is aryl or R¹⁰-substituted aryl;

Ar² is aryl or R¹¹-substituted aryl;

Q is —(CH₂)_{q}—, wherein q is 2-6, or, with the 3-position ring carbon of the azetidinone,

forms the spiro group

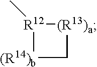

R¹² is

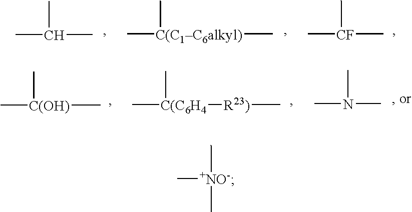

R¹³ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)═CH—; or R¹² together with an adjacent R¹³, or R¹² together with an adjacent R¹⁴, form a —CH═CH— or a —CH═C(C₁-C₆ alkyl)- group;

a and b are independently 0, 1, 2 or 3, provided both are not zero; provided that when R¹³ is —CH═CH— or —C(C₁-C₆ alkyl)═CH—, a is 1; provided that when R¹⁴ is —CH═CH— or —C(C₁-C₆ alkyl)═CH—, b is 1; provided that when a is 2 or 3, the R¹³'s can be the same or different; and provided that when b is 2 or 3, the R¹⁴'s can be the same or different;

R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;

Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —COOR¹⁹;

R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and

R²⁵ is H, —OH or (C₁-C₆)alkoxy.

Therapeutic combinations are provided comprising: (a) at least one of an HM74A or HM74 agonist (e.g., nicotinic acid or derivatives thereof); and (b) a second amount of at least one sterol absorption inhibitor represented by Formulae (I-XI), substituted azetidinone compounds, substituted β-lactam compounds, or pharmaceutically acceptable salts or solvates of the sterol absorption inhibitors, wherein the first amount and the second amount together comprise a therapeutically effective amount for the treatment or prevention of a vascular condition, diabetes, obesity or lowering a concentration of a sterol in plasma of a subject.

Pharmaceutical compositions for the treatment or prevention of a vascular condition, diabetes, obesity or lowering a concentration of a sterol in plasma of a subject, comprising a therapeutically effective amount of the above compositions or therapeutic combinations and a pharmaceutically acceptable carrier also are provided.

Methods of treating or preventing a vascular condition, diabetes, obesity or lowering a concentration of a sterol in plasma of a subject, comprising the step of administering to a subject in need of such treatment an effective amount of the above compositions or therapeutic combinations also are provided.

Other than in the operating examples, or where otherwise indicated, all numbers expressing quantities of ingredients, reaction conditions, and so forth used in the specification and claims are to be understood as being modified in all instances by the term “about.”

### DETAILED DESCRIPTION

In one embodiment, the present invention is directed to compositions, pharmaceutical compositions, therapeutic combinations, kits and methods of treatment using the same comprising at least one (one or more) HM74A or HM74 receptor agonists and at least one (one or more) of substituted azetidinone sterol absorption inhibitors or substituted β-lactam sterol absorption inhibitors such as are discussed in detail below.

Non-limiting examples of suitable HM74 and HM74A receptor agonists include nicotinic acid and derivatives thereof, 5-methylpyrazole-3-carboxylic acid and acifran (4,5-dihydro-5-methyl-4-oxo-5-phenyl-2-furan carboxylic acid). These compounds have been identified as agonists of the HM74A or HM74 receptor (Wise et al., J. Biol. Chem. (2003) 278(11): 9869-9874). The nucleotide sequence of human HM74A is disclosed under Genbank/EBI Data Bank Accession No. AY148884 and rat HM74A is disclosed under EMM_patAR098624.

As used herein, “nicotinic acid derivative” means a compound comprising a pyridine-3-carboxylate structure or a pyrazine-2-carboxylate structure, including acid forms, salts, esters, zwitterions and tautomers, where available. Examples of nicotinic acid derivatives include pyradine-3-acetic acid, 5-methyl nicotinic acid, nicotinuric acid, niceritrol, nicofuranose and acipimox (5-methylpyrazine-2-carboxylic acid 4-oxide). Nicotinic acid and its derivatives inhibit hepatic production of VLDL and its metabolite LDL and increases HDL and apo A-1 levels. An example of a suitable nicotinic acid product is NIASPAN® (niacin extended-release tablets) which are available from Kos.

One skilled in the art can very easily identify other substances (e.g., nicotinic acid derivatives) which bind to and agonize the HM74A or HM74 receptor. For example, Wise et al. (J. Biol. Chem (2003) 278(11):9869-9874) disclose a labeled nicotine binding assay as well as a [³⁵S]-GTPγS binding assay. Soga et al. (Biochem. Biophys. Res. Comm. (2003) 303:364-369) discloses another radiolabel binding assay using the HM74 receptor which could easily be adapted to the HM74A receptor. Tunaru et al. (Nature Medicine (2003) 9(3): 352-355) discloses a calcium mobilization assay using the HM74 receptor which, similarly, can be adapted to the HM74A receptor. Moreover, FLIPR assays are described generally in U.S. Pat. No. 6,420,183 and may be adapted to the HM74A or HM74 receptor.

“Subject” means any organism, preferably an animal, more preferably a mammal (e.g., dog, rabbit, mouse, rat, horse, cow, cat, guinea pig, hamster) and even more preferably a human.

These assays employ conventional laboratory methods which are commonly known in the art. Some of these techniques, for example, are explained in the literature. See, e.g., Sambrook, Fritsch & Maniatis, Molecular Cloninq: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

The HM74 and/or HM74A receptor agonists are administered in a therapeutically effective amount to treat the specified condition, for example a total daily dosage of HM74 and/or HM74A receptor agonists can range from about 500 to about 10,000 mg/day, preferably about 1000 to about 8000 mg/day, and more preferably about 3000 to about 6000 mg/day, given in a single dose or 2-4 divided doses. The exact dose, however, is determined by the attending clinician and is dependent on such factors as the potency of the compound administered, the age, weight, condition and response of the patient.

The phrase “therapeutically effective amount” means that amount of a therapeutic agent of the composition, such as the HM74 and/or HM74A receptor agonists, sterol absorption inhibitor(s) and pharmacological or therapeutic agents described below, that will elicit a biological or medical response of a tissue, system, subject, animal or mammal that is being sought by the administrator (such as a researcher, doctor or veterinarian) which includes alleviation of the symptoms of the condition or disease being treated and the prevention, slowing or halting of progression of one or more conditions, for example vascular conditions, such as hyperlipidemia (for example atherosclerosis, hypercholesterolemia or sitosterolemia), vascular inflammation, stroke, diabetes, obesity and/or to reduce the level of sterol(s) (such as cholesterol) in the plasma.

As used herein, “combination therapy” or “therapeutic combination” means the administration of two or more therapeutic agents, such as HM74 or HM74A receptor agonists and sterol absorption inhibitor(s), to prevent or treat a condition, for example a vascular condition, such as hyperlipidaemia (for example atherosclerosis, hypercholesterolemia or sitosterolemia), stroke, diabetes, obesity and/or reduce the level of sterol(s) in the plasma. As used herein, “vascular” comprises cardiovascular, cerebrovascular, peripheral vascular and combinations thereof. Such administration includes coadministration of these therapeutic agents in a substantially simultaneous manner, such as in a single tablet or capsule having a fixed ratio of active ingredients or in multiple, separate capsules for each therapeutic agent. Also, such administration includes use of each type of therapeutic agent in a sequential manner. In either case, the treatment using the combination therapy will provide beneficial effects in treating the condition. A potential advantage of the combination therapy disclosed herein may be a reduction in the required amount of an individual therapeutic compound or the overall total amount of therapeutic compounds that are effective in treating the condition. By using a combination of therapeutic agents, the side effects of the individual compounds can be reduced as compared to a monotherapy, which can improve patient compliance. Also, therapeutic agents can be selected to provide a broader range of complementary effects or complimentary modes of action.

As discussed above, the compositions, pharmaceutical compositions and therapeutic combinations of the present invention comprise one or more substituted azetidinone or substituted β-lactam sterol or 5α-stanol absorption inhibitors discussed in detail below. As used herein, “sterol absorption inhibitor” means a compound capable of inhibiting the absorption of one or more sterols, including but not limited to cholesterol, phytosterols (such as sitosterol, campesterol, stigmasterol and avenosterol) and mixtures thereof, when administered in a therapeutically effective (sterol absorption inhibiting) amount to a subject. As used herein, “5α-stanol absorption inhibitor” means a compound capable of inhibiting the absorption of one or more 5α-stanols, including, but not limited to, cholestanol, 5α-campestanol, and 5α-sitostanol and mixtures thereof, when administered in a therapeutically effective (5α-stanol absorption inhibiting) amount to a subject.

In a preferred embodiment, sterol absorption inhibitors useful in the compositions, therapeutic combinations and methods of the present invention are represented by Formula (I):

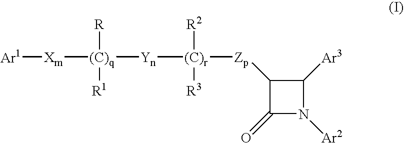

or isomers of the compounds of Formula (I), or pharmaceutically acceptable salts or solvates of the compounds of Formula (I) or of the isomers of the compounds of Formula (I) as discussed in detail above.

As used herein, the term “alkyl” or “lower alkyl” means straight or branched alkyl chains having from 1 to 6 carbon atoms and “alkoxy” means alkoxy groups having 1 to 6 carbon atoms. Non-limiting examples of lower alkyl groups include, for example methyl, ethyl, propyl, and butyl groups.

“Alkenyl” means straight or branched carbon chains having one or more double bonds in the chain, conjugated or unconjugated. Similarly, “alkynyl” means straight or branched carbon chains having one or more triple bonds in the chain. Where an alkyl, alkenyl or alkynyl chain joins two other variables and is therefore bivalent, the terms alkylene, alkenylene and alkynylene are used.

“Cycloalkyl” means a saturated carbon ring of 3 to 6 carbon atoms, while “cycloalkylene” refers to a corresponding bivalent ring, wherein the points of attachment to other groups include all positional isomers.

“Halogeno” refers to fluorine, chlorine, bromine or iodine radicals.

“Aryl” means phenyl, naphthyl, indenyl, tetrahydronaphthyl or indanyl.

“Phenylene” means a bivalent phenyl group, including ortho, meta and para-substitution.

The statements wherein, for example, R, R¹, R² and R³, are said to be independently selected from a group of substituents, mean that R, R¹, R² and R³ are independently selected, but also that where an R, R¹, R² and R³ variable occurs more than once in a molecule, each occurrence is independently selected (e.g., if R is —OR⁶, wherein R⁶ is hydrogen, R² can be —OR⁶ wherein R⁶ is lower alkyl). Those skilled in the art will recognize that the size and nature of the substituent(s) will affect the number of substituents that can be present.

Compounds of the invention have at least one asymmetrical carbon atom and therefore all isomers, including enantiomers, stereoisomers, rotamers, tautomers and racemates of the compounds of Formulae (I-XI) (where they exist) are contemplated as being part of this invention. The invention includes d and I isomers in both pure form and in admixture, including racemic mixtures. Isomers can be prepared using conventional techniques, either by reacting optically pure or optically enriched starting materials or by separating isomers of a compound of the Formulae I-XI. Isomers may also include geometric isomers, e.g., when a double bond is present.

Those skilled in the art will appreciate that for some of the compounds of the Formulas I-XI, one isomer will show greater pharmacological activity than other isomers.

Compounds of the invention with an amino group can form pharmaceutically acceptable salts with organic and inorganic acids. Examples of suitable acids for salt formation are hydrochloric, sulfuric, phosphoric, acetic, citric, oxalic, malonic, salicylic, malic, fumaric, succinic, ascorbic, maleic, methanesulfonic and other mineral and carboxylic acids well known to those in the art. The salt is prepared by contacting the free base form with a sufficient amount of the desired acid to produce a salt. The free base form may be regenerated by treating the salt with a suitable dilute aqueous base solution such as dilute aqueous sodium bicarbonate. The free base form differs from its respective salt form somewhat in certain physical properties, such as solubility in polar solvents, but the salt is otherwise equivalent to its respective free base forms for purposes of the invention.

Certain compounds of the invention are acidic (e.g., those compounds which possess a carboxyl group). These compounds form pharmaceutically acceptable salts with inorganic and organic bases. Examples of such salts are the sodium, potassium, calcium, aluminum, gold and silver salts. Also included are salts formed with pharmaceutically acceptable amines such as ammonia, alkyl amines, hydroxyalkylamines, N-methylglucamine and the like.

As used herein, “solvate” means a molecular or ionic complex of molecules or ions of solvent with those of solute (for example, one or more compounds of Formulae I-XI, isomers of the compounds of Formulae I-XI, or prodrugs of the compounds of Formulae I-XI). Non-limiting examples of useful solvents include polar, protic solvents such as water and/or alcohols (for example methanol).

As used herein, “prodrug” means' compounds that are drug precursors which, following administration to a patient, release the drug in vivo via some chemical or physiological process (e.g., a prodrug on being brought to the physiological pH or through enzyme action is converted to the desired drug form).

Preferred compounds of Formula (I) are those in which Ar is phenyl or R substituted phenyl, more preferably (4-R⁴)-substituted phenyl. Ar² is preferably phenyl or R⁴-substituted phenyl, more preferably (4-R⁴)-substituted phenyl. Ar³ is preferably R⁵-substituted phenyl, more preferably (4-R⁵)-substituted phenyl. When Ar¹ is (4-R⁴)-substituted phenyl, R⁴ is preferably a halogen. When Ar² and Ar³ are R⁴- and R⁵-substituted phenyl, respectively, R⁴ is preferably halogen or —OR⁶ and R⁵ is preferably —OR⁶, wherein R⁶ is lower alkyl or hydrogen. Especially preferred are compounds wherein each of Ar¹ and Ar² is 4-fluorophenyl and Ar³ is 4-hydroxyphenyl or 4-methoxyphenyl.

X, Y and Z are each preferably —CH₂—. R¹ and R³ are each preferably hydrogen. R and R² are preferably —OR⁶ herein R⁶ is hydrogen, or a group readily metabolizable to a hydroxyl (such as —O(CO)R⁶, —O(CO)OR⁹ and —O(CO)NR⁶R⁷, defined above).

The sum of m, n, p, q and r is preferably 2, 3 or 4, more preferably 3. Preferred are compounds wherein m, n and r are each zero, q is 1 and p is 2.

Also preferred are compounds of Formula (I) in which p, q and n are each zero, r is 1 and m is 2 or 3. More preferred are compounds wherein m, n and r are each zero, q is 1, p is 2, Z is —CH₂— and R is —OR⁶, especially when R⁶ is hydrogen.

Also more preferred are compounds of Formula (I) wherein p, q and n are each zero, r is 1, m is 2, X is —CH₂— and R² is —OR⁶, especially when R⁶ is hydrogen.

Another group of preferred compounds of Formula (I) is that in which Ar¹ is phenyl or R⁴-substituted phenyl, Ar² is phenyl or R⁴-substituted phenyl and Ar³ is R⁵-substituted phenyl. Also preferred are compounds in which Ar¹ is phenyl or R⁴-substituted phenyl, Ar² is phenyl or R⁴-substituted phenyl, Ar³ is R⁵-substituted phenyl, and the sum of m, n, p, q and r is 2, 3 or 4, more preferably 3. More preferred are compounds wherein Ar¹ is phenyl or R⁴-substituted phenyl, Ar² is phenyl or R⁴-substituted phenyl, Ar³ is R⁵-substituted phenyl, and wherein m, n and r are each zero, q is 1 and p is 2, or wherein p, q and n are each zero, r is 1 and m is 2 or 3.

In the compound of Formula I, preferably, R⁴ is 1-3 independently selected substituents, and R⁵ is preferably 1-3 independently selected substituents.

In a preferred embodiment, a sterol inhibitor of Formula (I) (ezetimibe) useful in the compositions, therapeutic combinations and methods of the present invention is represented by Formula (II) below:

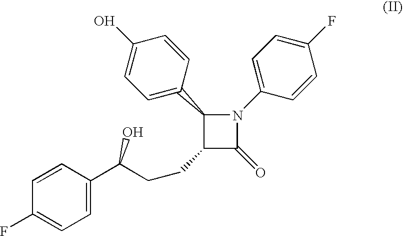

or pharmaceutically acceptable salts or solvates of the compound of Formula (II).

Compounds of Formula I can be prepared by a variety of methods well know to those skilled in the art, for example such as are disclosed in U.S. Pat. Nos. 5,631,365, 5,767,115, 5,846,966, 6,207,822 and U.S. Patent Publication No. 2002/0193607, and PCT Patent Application WO 93/02048, each of which is incorporated herein by reference, and in the Example below.

Preferred compounds of Formula (III) include those in which Ar¹ is R³-substituted phenyl, especially (4-R³) substituted phenyl. Ar² is preferably R⁴-substituted phenyl, especially (4-R⁴)-substituted phenyl. Ar³ is preferably R⁵-substituted phenyl, especially (4-R⁵)-substituted phenyl. Mono-substitution of each of Ar¹, Ar² and Ar³ is preferred.

Y and Z are each preferably —CH₂—. R² is preferably hydrogen. R¹ is preferably —OR⁶ wherein R⁶ is hydrogen, or a group readily metabolizable to a hydroxyl (such as —O(CO)R⁶, —O(CO)OR⁹ and —O(CO)NR⁶R⁷, defined above). Also preferred are compounds wherein R¹ and R² together are ═O.

The sum of q and p is preferably 1 or 2, more preferably 1. Preferred are compounds wherein p is zero and q is 1. More preferred are compounds wherein p is zero, q is 1, Y is —CH₂— and R is —OR⁶, especially when R⁶ is hydrogen.

Another group of preferred compounds of Formula (III) is that in which Ar¹ is R³-substituted phenyl, Ar² is R⁴-substituted phenyl and Ar³ is R⁵-substituted phenyl.

Also preferred are compounds of Formula (III) wherein Ar¹ is R³-substituted phenyl, Ar² is R⁴-substituted phenyl, Ar³ is R⁵-substituted phenyl, and the sum of p and q is 1 or 2, especially 1. More preferred are compounds wherein Ar¹ is R³-substituted phenyl, Ar² is R⁴-substituted phenyl, Ar³ is R⁵-substituted phenyl, p is zero and q is 1.

A is preferably —O—.

R³ is preferably —COOR⁶, —CONR⁶R⁷, —COR⁶, —SO₂NR⁶R⁷, S(O)₀₋₂-alkyl, S(O)₀₋₂-aryl, NO₂ or halogeno. A more preferred definition for R³ is halogeno, especially fluoro or chloro.

R⁴ is preferably hydrogen, lower alkyl, —OR⁶, —O(CO)R⁶, —O(CO)OR⁹, —O(CO)NR⁶R⁷, —NR⁶R⁷, COR⁶ or halogeno, wherein R⁶ and R⁷ are preferably independently hydrogen or lower alkyl, and R⁹ is preferably lower alkyl. A more preferred definition for R⁴ is hydrogen or halogeno, especially fluoro or chloro.

R⁵ is preferably —OR⁶, —O(CO)R⁶, —O(CO)OR⁹, —O(CO)NR⁶R⁷, —NR⁶R⁷, -(lower alkylene)-COOR⁶ or —CH═CH—COOR⁶, wherein R⁶ and R⁷ are preferably independently hydrogen or lower alkyl, and R⁹ is preferably lower alkyl. A more preferred definition for R⁵ is —OR⁶, -(lower alkylene)-COOR⁶ or —CH═CH—COOR⁶, wherein R⁶ is preferably hydrogen or lower alkyl.

Methods for making compounds of Formula III are well known to those skilled in the art. Non-limiting examples of suitable methods are disclosed in U.S. Pat. No. 5,688,990, which is incorporated herein by reference.

As used in Formula (IV) above, “A” is preferably an R²-substituted, 6-membered heterocycloalkyl ring containing 1 or 2 nitrogen atoms. Preferred heterocycloalkyl rings are piperidinyl, piperazinyl and morpholinyl groups. The ring “A” is preferably joined to the phenyl ring through a ring nitrogen. Preferred R² substituents are hydrogen and lower alkyl. R¹⁹ is preferably hydrogen.

Ar² is preferably phenyl or R⁴-phenyl, especially (4-R⁴)-substituted phenyl. Preferred definitions of R⁴ are lower alkoxy, especially methoxy, and halogeno, especially fluoro.

Ar¹ is preferably phenyl or R³-substituted phenyl, especially (4-R³)-substituted phenyl.

There are several preferred definitions for the —R¹-Q- combination of variables:

Q is a bond and R¹ is lower alkylene, preferably propylene;

Q is a spiro group as defined above, wherein preferably R⁶ and R⁷ are each ethylene and R⁵ is

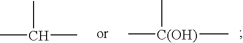

Q is a bond and R¹ is

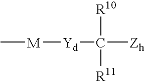

wherein the variables are chosen such that R¹ is —O—CH₂—CH(OH)—;

Q is a bond and R¹ is

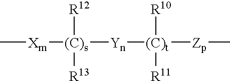

wherein the variables are chosen such that R¹ is —CH(OH)—(CH₂)₂—; and

Q is a bond and R¹ is

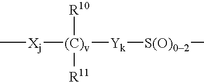

wherein the variables are chosen such that R¹ is —CH(OH)—CH₂—S(O)₀₋₂—.

Methods for making compounds of Formula IV are well known to those skilled in the art. Non-limiting examples of suitable methods are disclosed in U.S. Pat. No. 5,656,624, which is incorporated herein by reference.

Within the scope of Formula V, there are included two preferred structures. In Formula VA, q is zero and the remaining variables are as defined above, and in Formula VB, q is 1 and the remaining variables are as defined above:

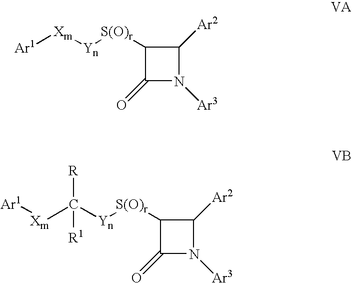

R⁴, R⁵ and R¹⁰ are each preferably 1-3 independently selected substituents as set forth above. Preferred are compounds of Formula (V) wherein Ar¹ is phenyl, R¹⁰-substituted phenyl or thienyl, especially (4-R¹⁰)-substituted phenyl or thienyl. Ar² is preferably R⁴-substituted phenyl, especially (4-R⁴)-substituted phenyl. Ar³ is preferably phenyl or R⁵-substituted phenyl, especially (4-R²)-substituted phenyl. When Ar¹ is R¹⁰-substituted phenyl, R¹⁰ is preferably halogeno, especially fluoro. When Ar² is R⁴-substituted phenyl, R⁴ is preferably —OR⁶, especially wherein R⁶ is hydrogen or lower alkyl. When Ar³ is R⁵-substituted phenyl, R⁵ is preferably halogeno, especially fluoro. Especially preferred are compounds of Formula (V) wherein Ar¹ is phenyl, 4-fluorophenyl or thienyl, Ar² is 4-(alkoxy or hydroxy)phenyl, and Ar³ is phenyl or 4-fluorophenyl.

X and Y are each preferably —CH₂—. The sum of m, n and q is preferably 2, 3 or 4, more preferably 2. When q is 1, n is preferably 1 to 5.

Preferences for X, Y, Ar¹, Ar² and Ar³ are the same in each of Formulae (VA) and (VB).

In compounds of Formula (VA), the sum of m and n is preferably 2, 3 or 4, more preferably 2. Also preferred are compounds wherein the sum of m and n is 2, and r is 0 or 1.

In compounds of Formula (VB), the sum of m and n is preferably 1, 2 or 3, more preferably 1. Especially preferred are compounds wherein m is zero and n is 1. R¹ is preferably hydrogen and R is preferably —OR⁶ wherein R⁶ is hydrogen, or a group readily metabolizable to a hydroxyl (such as —O(CO)R⁶, —O(CO)OR⁹ and —O(CO)NR⁶R⁷, defined above), or R and R¹ together form a ═O group.

Methods for making compounds of Formula V are well known to those skilled in the art. Non-limiting examples of suitable methods are disclosed in U.S. Pat. No. 5,624,920, which is incorporated herein by reference.

One group of preferred compounds of Formula VI is that in which R₂₁ is selected from phenyl, W-substituted phenyl, indanyl, benzofuranyl, benzodioxolyl, tetrahydronaphthyl, pyridyl, pyrazinyl, pyrimidinyl, quinolyl or cyclopropyl,

wherein W is lower alkyl, lower alkoxy, OH, halogeno, —N(R₈)(R₉), —NHC(O)OR₁₀, —NHC(O)R₁₀, NO₂, —CN, —N₃, —SH, —S(O)₀₋₂-(lower alkyl), —COOR₁₉, —CON(R₈)(R₉), —COR₁₂, phenoxy, benzyloxy, —OCF₃, —CH═C(O)R₁₂ or tert-butyldimethylsilyloxy, wherein R₈, R₉, R₁₀, R₁₂ and R₁₉ are as defined for Formula IV. When W is 2 or 3 substituents, the substituents can be the same or different.

Another group of preferred compounds of Formula VI is that in which R₂₀ is phenyl or W-substituted phenyl, wherein preferred meanings of W are as defined above for preferred definitions of R₂₁.

More preferred are compounds of Formula VI wherein R₂₀ is phenyl or W-substituted phenyl and R₂₁ is phenyl, W-substituted phenyl, indanyl, benzofuranyl, benzodioxolyl, tetrahydronaphthyl, pyridyl, pyrazinyl, pyrimidinyl, quinolyl or cyclopropyl; W is lower alkyl, lower alkoxy, OH, halogeno, —N(R₈)(R₉), —NHC(O)OR₁₀, —NHC(O)R₁₀, NO₂, —CN, —N₃, —SH, —S(O)₀₋₂-(lower alkyl), —COOR₁₉, —CON(R₈)(R₉), —COR₁₂, phenoxy, benzyloxy, —CH═CHC(O)R₁₂, —OCF₃ or tert-butyl-dimethyl-silyloxy, wherein when W is 2 or 3 substituents, the substituents can be the same or different, and wherein R₈, R₉, R₁₀, R₁₂ and R₁₉ are as defined in Formula VI.

Also preferred are compounds of Formula VI wherein R₁ is

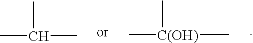

Another group of preferred compounds of Formula VI is in which R₂ and R₃ are each —CH₂— and the sum of u and v is 2, 3 or 4, with u=v=2 being more preferred.

R₄ is preferably B—(CH₂)_{q}— or B—(CH₂)ₑ-Z-(CH₂)ᵣ—, wherein B, Z, q, e and r are as defined above. B is preferably

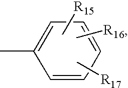

wherein R₁₆ and R₁₇ are each hydrogen and wherein R₁₅ is preferably H, OH, lower alkoxy, especially methoxy, or halogeno, especially chloro.

Preferably Z is —O—, e is 0, and r is 0.

Preferably q is 0-2.

R₂₀ is preferably phenyl or W-substituted phenyl.

Preferred W substituents for R₂₀ are lower alkoxy, especially methoxy and ethoxy, OH, and —C(O)R₁₂, wherein R₁₂ is preferably lower alkoxy.

Preferably R₂₁ is selected from phenyl, lower alkoxy-substituted phenyl and F-phenyl.

Especially preferred are compounds of Formula VI wherein R₁ is

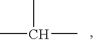

or

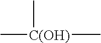

R₂ and R₃ are each —CH₂—, u=v=2, R₄ is B—(CH₂)_{q}—, wherein B is phenyl or phenyl substituted by lower alkoxy or chloro, q is 0-2, R₂₀ is phenyl, OH-phenyl, lower alkoxy-substituted phenyl or lower alkoxycarbonyl-substituted phenyl, and R₂₁ is phenyl, lower alkoxy-substituted phenyl or F-phenyl.

Methods for making compounds of Formula VI are well known to those skilled in the art. Non-limiting examples of suitable methods are disclosed in U.S. Pat. No. 5,698,548, which is incorporated herein by reference.

Preferred are compounds of Formula (VIIA) wherein R is hydrogen, saturated or mono-unsaturated C₁-C₁₀ alkyl or phenyl. Another group of preferred compounds of Formula (VIIA) is that in which D is propyl (i.e., —(CH₂)_{q}— and q is 3). A third group of preferred compounds of Formula (VIIA) is that wherein R₄ is p-methoxyphenyl or 2,4,6-trimethoxyphenyl. Still another group of preferred compounds of Formula (VIIA) is that wherein A is ethylene or a bond (i.e., —(CH₂)ₚ— wherein p is zero). R_{1′}, R_{2′}, and R_{3′} are preferably each hydrogen, and preferably R₁ is hydrogen, hydroxy, nitro, lower alkoxy, amino or t-butoxycarbonyl-amino and R₂ and R₃ are each hydrogen.

More preferred are compounds of Formula (VIIA) wherein R_{1′}, R_{2′}, and R_{3′} are each hydrogen; R₁ is hydrogen, hydroxy, nitro, lower alkoxy, amino or t-butoxycarbonyl-amino and R₂ and R₃ are each hydrogen; R is hydrogen, ethyl or phenyl; D is propyl; R₄ is p-methoxyphenyl or 2,4,6-trimethoxyphenyl; and A is ethylene or a bond.

Preferred compounds of Formula (VIIA), wherein B′ is phenyl, are shown in the following table:

| D | R | A | B | R₄ |
|---|---|---|---|---|
| —(CH₂)₃— | H | — | p-MeO- | p-MeO-phenyl |
| | | | phenyl | |
| —CH₂C(O)— | phenyl | — | phenyl | p-MeO-phenyl |
| —(CH₂)₃— | H | — | phenyl | p-MeO-phenyl |
| —(CH₂)₃— | H | — | p-OH- | p-MeO-phenyl |
| | | | phenyl | |
| —(CH₂)₃— | H | ethylene | p-MeO- | p-MeO-phenyl |
| | | | phenyl | |
| —(CH₂)₃— | H | — | 3-MeO- | p-MeO-phenyl |
| | | | phenyl | |
| —(CH₂)₃— | ethyl | — | phenyl | p-MeO-phenyl |
| —(CH₂)₃— | phenyl | — | phenyl | p-MeO-phenyl |
| —(CH₂)₃— | ethyl | — | phenyl | 2,4,6-tri-MeO- |
| | | | | phenyl |
| —(CH₂)₃— | methyl | — | phenyl | p-MeO-phenyl |
| —(CH₂)₃— | H | — | p-NH₂- | p-MeO-phenyl |
| | | | phenyl | |

The first-listed compound in the above table having the (3R,4S) absolute stereochemistry is more preferred.

Preferred compounds of Formula (VIIB) are those wherein R is hydrogen, methyl, ethyl, phenyl or phenylpropyl. Another group of preferred compounds of Formula (VIIB) is that wherein R₄ is p-methoxyphenyl or 2,4,6-trimethoxyphenyl. Still another group of preferred compounds of Formula (VIB) is that wherein A is ethylene or a bond. Yet another group of preferred compounds of Formula (VIIB) is that wherein E is decyl, oleoyl or 7-Z-hexadecenyl. Preferably R₁, R₂ and R₃ are each hydrogen.

More preferred compounds of Formula (VIIB) are those wherein R is hydrogen, methyl, ethyl, phenyl or phenylpropyl; R₄ is p-methoxyphenyl or 2,4,6-trimethoxyphenyl; A is ethylene or a bond; E is decyl, oleoyl or 7-Z-hexadecenyl; and R₁, R₂ and R₃ are each hydrogen.

A preferred compound of Formula (VIIB) is that wherein E is decyl, R is hydrogen, B-A is phenyl and R₄ is p-methoxyphenyl.

In the compound of Formula (VIII), Ar² is preferably phenyl or R¹¹-phenyl, especially (4-R¹¹)-substituted phenyl. Preferred definitions of R¹¹ are lower alkoxy, especially methoxy, and halogeno, especially fluoro.

Ar¹ is preferably phenyl or R¹⁰-substituted phenyl, especially (4-R¹⁰)-substituted phenyl. Preferably R¹⁰ is halogeno, and more preferably fluoro.

There are several preferred definitions for the —R¹-Q- combination of variables:

Q is a bond and R¹ is lower alkylene, preferably propylene;

Q is a spiro group as defined above, wherein preferably R¹³ and R¹⁴ are each ethylene and R¹² is

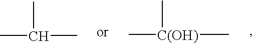

and R¹ is —(CH₂)_{q} wherein q is 0-6;

Q is a bond and R¹ is

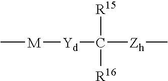

wherein the variables are chosen such that R¹ is —O—CH₂—CH(OH)—;

Q is a bond and R¹

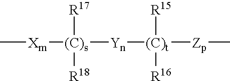

wherein the variables are chosen such that R¹ is —CH(OH)—(CH₂)₂—; and

Q is a bond and R¹ is

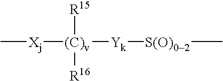

wherein the variables are chosen such that R¹ is —CH(OH)—CH₂—S(O)₀₋₂—.

A preferred compound of Formula (VIII) therefore, is one wherein G and G¹ are as defined above and in which the remaining variables have the following definitions:

Ar¹ is phenyl or R¹⁰-substituted phenyl, wherein R¹⁰ is halogeno;

Ar² is phenyl or R¹¹-phenyl, wherein R¹¹ is 1 to 3 substituents independently selected from the group consisting of C₁-C₆ alkoxy and halogeno;

Q is a bond and R¹ is lower alkylene; Q, with the 3-position ring carbon of the azetidinone, forms the group

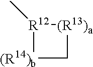

wherein preferably R¹³ and R¹⁴ are each ethylene and a and b are each 1, and wherein R¹² is

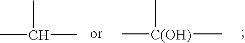

Q is a bond and R¹ is —O—CH₂—CH(OH)—; Q is a bond and R¹ is —CH(OH)—(CH₂)₂—; or Q is a bond and R¹ is —CH(OH)—CH₂—S(O)₀₋₂—.

Preferred variables for G and G¹ groups of the formulae

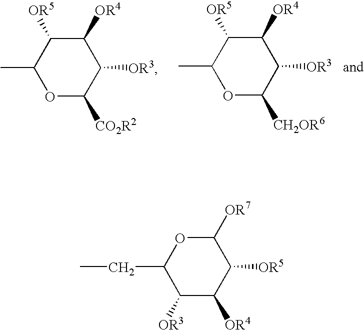

are as follows:

R², R³, R⁴, R⁵, R⁶ and R⁷ are independently selected from the group consisting of H, (C₁-C₆)alkyl, benzyl and acetyl.

Preferred variables for group G or G¹ of the formula:

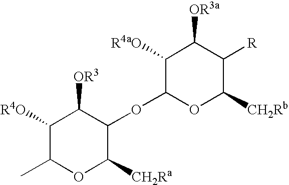

are as follows:

R³, R^{3a}, R⁴ and R^{4a} are selected from the group consisting of H, (C₁-C₆)alkyl, benzyl and acetyl;

R, R^{a} and R^{b} are independently selected from the group consisting of H, —OH, halogeno, —NH₂, azido, (C₁-C₆)alkoxy(C₁-C₆)alkoxy and —W—R³⁰,

wherein W is —O—C(O)— or —O—C(O)—NR³¹—, R³¹ is H and R³⁰ is (C₁-C₆)alkyl, —C(O)—(C₁-C₄)alkoxy-(C₁-C₆)alkyl, T, T-(C₁-C₆)alkyl, or T or T-(C₁-C₆)alkyl wherein T is substituted by one or two halogeno or (C₁-C₆)alkyl groups.

Preferred R³⁰ substituents are selected from the group consisting of: 2-fluorophenyl, 2,4-difluoro-phenyl, 2,6-dichlorophenyl, 2-methylphenyl, 2-thienylmethyl, 2-methoxy-carbonylethyl, thiazol-2-yl-methyl, 2-furyl, 2-methoxycarbonylbutyl and phenyl.

Preferred combinations of R, R^{a} and R^{b} are as follows:

1) R, R^{a} and R^{b} are independently —OH or —O—C(O)—NH—R³⁰, especially wherein R^{a} is —OH and R and R^{b} are —O—C(O)—NH—R³⁰ and R³⁰ is selected from the preferred substituents identified above, or wherein R and R^{a} are each —OH and R^{b} is —O—C(O)—NH—R³⁰ wherein R³⁰ is 2-fluorophenyl, 2,4-difluorophenyl, 2,6-dichlorophenyl;

2) R^{a} is —OH, halogeno, azido or (C₁-C₆)-alkoxy(C₁-C₆)alkoxy, R^{b} is H, halogeno, azido or (C₁-C₆)alkoxy(C₁-C₆)-alkoxy, and R is —O—C(O)—NH—R³⁰, especially compounds wherein R^{a} is —OH, R^{b} is H and R³⁰ is 2-fluorophenyl;

3) R, R^{a} and R^{b} are independently —OH or —O—C(O)—R³⁰ and R³⁰ is (C₁-C₆)alkyl, T, or T substituted by one or two halogeno or (C₁-C₆)alkyl groups, especially compounds wherein R is —OH and R^{a} and R^{b} are —O—C(O)—R³⁰ wherein R³⁰ is 2-furyl; and

4) R, R^{a} and R^{b} are independently —OH or halogeno. Three additional classes of preferred compounds are those wherein the C^{1′} anomeric oxy is beta, wherein the C^{2′} anomeric oxy is beta, and wherein the R group is alpha. G and G¹ are preferably selected from:

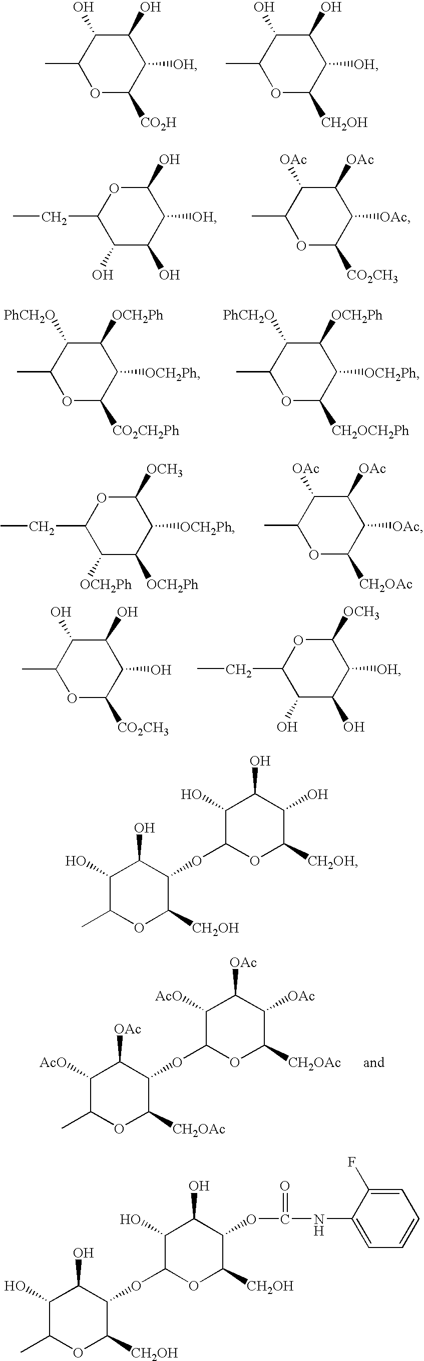

wherein Ac is acetyl and Ph is phenyl.

Preferably, R²⁶ is H or OH, more preferably H. The —O-G substituent is preferably in the 4-position of the phenyl ring to which it is attached.

In Formula (IX), Ar² is preferably phenyl or R¹¹-phenyl, especially (4-R¹¹)-substituted phenyl. Preferred definitions of R¹¹ are lower alkoxy, especially methoxy, and halogeno, especially fluoro.

Ar¹ is preferably phenyl or R¹⁰-substituted phenyl, especially (4-R¹⁰)-substituted phenyl. A preferred definition of R¹⁰ is halogeno, especially fluoro.

Preferably Q is a lower alkyl or a spiro group as defined above, wherein preferably R¹³ and R¹⁴ are each ethylene and R¹² is

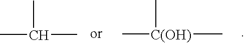

A preferred compound of formula IX, therefore, is one wherein R¹ is as defined above and in which the remaining variables have the following definitions:

Ar¹ is phenyl or R¹⁰-substituted phenyl, wherein R¹⁰ is halogeno;

Ar² is phenyl or R¹¹-phenyl, wherein R¹¹ is 1 to 3 substituents independently selected from the group consisting of C₁-C₆ alkoxy and halogeno;

Q is a lower alkyl (i.e. C-1 to C-2) with Q=C-2 being preferred, or Q, with the

3-position ring carbon of the azetidinone, forms the group

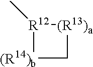

wherein preferably R¹³ and R¹⁴ are each ethylene and a and b are each 1, and wherein R¹² is

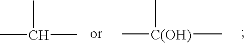

Preferred variables for R¹ groups of the formula

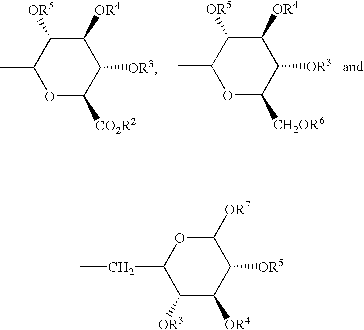

are as follows:

R², R³, R⁴, R⁵, R⁶ and R⁷ are independently selected from the group consisting of H, (C₁-C₆)alkyl, benzyl and acetyl.

Preferred variables for group R¹ of the formula

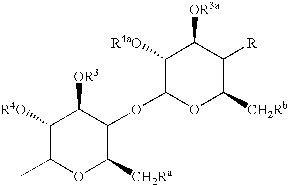

are as follows:

R³, R^{3a}, R⁴ and R^{4a} are selected from the group consisting of H, (C₁-C₆)alkyl, benzyl and acetyl;

R, R^{a} and R^{b} are independently selected from the group consisting of H, —OH, halogeno, —NH₂, azido, (C₁-C₆)alkoxy(C₁-C₆)alkoxy and —W—R³⁰, wherein W is —O—C(O)— or —O—C(O)—NR³¹—, R³¹ is H and R³⁰ is (C₁-C₆)alkyl, —C(O)—(C₁-C₄)alkoxy-(C₁-C₆)alkyl, T, T-(C₁-C₆)alkyl, or T or T-(C₁-C₆)alkyl wherein T is substituted by one or two halogeno or (C₁-C₆)alkyl groups.

Preferred R³⁰ substituents are 2-fluorophenyl, 2,4-difluoro-phenyl, 2,6-dichlorophenyl, 2-methylphenyl, 2-thienylmethyl, 2-methoxy-carbonylethyl, thiazol-2-yl-methyl, 2-furyl, 2-methoxycarbonylbutyl and phenyl. Preferred combinations of R, R^{a} and R^{b} are as follows: (1) R, R^{a} and R^{b} are independently —OH or —O—C(O)—NH—R³⁰, especially wherein R^{a} is —OH and R and R^{b} are —O—C(O)—NH—R³⁰ and R³⁰ is selected from the preferred substituents identified above, or wherein R and R^{a} are —OH and R^{b} is —O—C(O)—NH—R³⁰ wherein R³⁰ is 2-fluorophenyl, 2,4-difluoro-phenyl, 2,6-dichlorophenyl; (2) R^{a} is —OH, halogeno, azido or (C₁-C₆)-alkoxy(C₁-C₆)alkoxy, R^{b} is H, halogeno, azido or (C₁-C₆)alkoxy(C₁-C₆)-alkoxy, and R is —O—C(O)—NH—R³⁰, especially compounds wherein R^{a} is —OH, R^{b} is H and R³⁰ is 2-fluorophenyl; (3) R, R^{a} and R^{b} are independently —OH or —O—C(O)—R³⁰ and R³⁰ is (C₁-C₆)alkyl, T, or T substituted by one or two halogeno or (C₁-C₆)alkyl groups, especially compounds wherein R is —OH and R^{a} and R^{b} are —O—C(O)—R³⁰ wherein R³⁰ is 2-furyl; and (4) R, R^{a} and R^{b} are independently —OH or halogeno. Three additional classes of preferred are compounds are those wherein the C^{1′} anomeric oxy is beta, wherein the C^{2′} anomeric oxy is beta, and wherein the R group is alpha.

R¹ is preferably selected from:

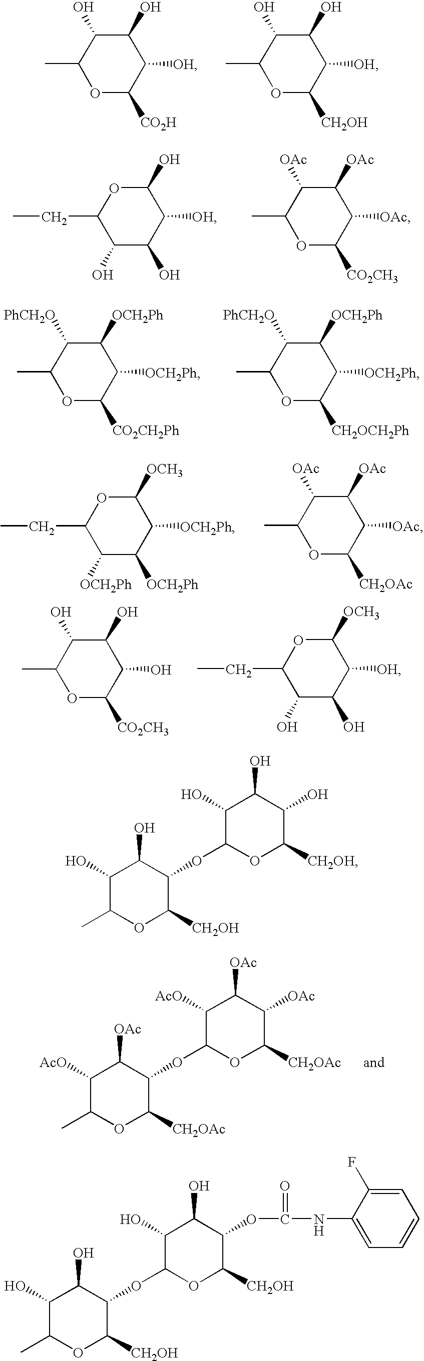

wherein Ac is acetyl and Ph is phenyl.

An example of a useful compound of this invention is one represented by the formula X:

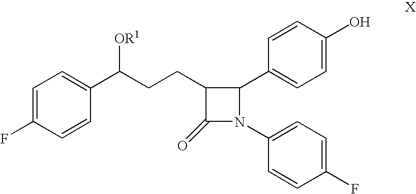

or pharmaceutically acceptable salts or solvates of the compound of Formula (X).

A more preferred compound is one represented by formula XI:

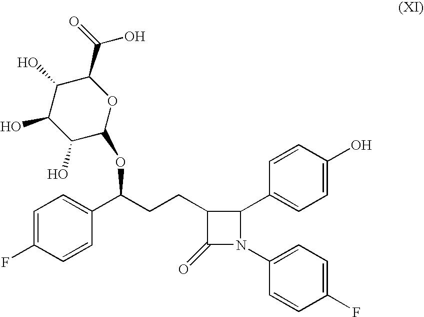

or pharmaceutically acceptable salts or solvates of the compound of Formula (XI).

In another embodiment, compositions, pharmaceutical compositions, therapeutic combinations, kits and methods of treatment as described above are provided which comprise: (a) a first amount of at least one HM74 or HM74A receptor agonist; and (b) a second amount of at least one substituted azetidinone compound or at least one substituted β-lactam compound, or isomers of the at least one substituted azetidinone compound or the at least one substituted β-lactam compound, or pharmaceutically acceptable salts or solvates of the at least one substituted azetidinone compound or the at least one substituted β-lactam compound or of the isomers of the at least one substituted azetidinone compound or the at least one substituted β-lactam compound, wherein the first amount and the second amount together in their totality (whether administered concurrently or consecutively) comprise a therapeutically effective amount for the treatment or prevention of a vascular condition, diabetes, obesity or lowering a concentration of a sterol in plasma of a subject. Suitable substituted azetidinone compounds or substituted β-lactam compounds can be selected from any of the compounds discussed above in Formulae I-XI. Other useful substituted azetidinone compounds include N-sulfonyl-2-azetidinones such as are disclosed in U.S. Pat. No. 4,983,597 and ethyl 4-(2-oxoazetidin-4-yl)phenoxy-alkanoates such as are disclosed in Ram et al., Indian J. Chem. Sect. B. 29B, 12 (1990), p.1134-7, which are incorporated by reference herein.

The compounds of Formulae I-XI can be prepared by known methods, including the methods discussed above and, for example, WO 93/02048 describes the preparation of compounds wherein —R¹-Q- is alkylene, alkenylene or alkylene interrupted by a hetero atom, phenylene or cycloalkylene; WO 94/17038 describes the preparation of compounds wherein Q is a spirocyclic group; WO 95/08532 describes the preparation of compounds wherein —R¹-Q- is a hydroxy-substituted alkylene group; PCT/US95/03196 describes compounds wherein —R¹-Q- is a hydroxy-substituted alkylene attached to the Ar¹ moiety through an —O— or S(O)₀-2-group; and U.S. Ser. No. 08/463,619, filed Jun. 5, 1995, describes the preparation of compounds wherein —R¹-Q- is a hydroxy-substituted alkylene group attached the azetidinone ring by a —S(O)₀₋₂- group.

The daily dose of the sterol or 5α-stanol absorption inhibitor(s) preferably ranges from about 0.1 to about 1000 mg per day, and more preferably about 0.25 to about 50 mg/day, given in a single dose or 2-4 divided doses. The exact dose, however, is determined by the attending clinician and is dependent on the potency of the compound administered, the age, weight, condition and response of the patient.

For administration of pharmaceutically acceptable salts of the above compounds, the weights indicated above refer to the weight of the acid equivalent or the base equivalent of the therapeutic compound derived from the salt.

In one embodiment of the present invention, the compositions or therapeutic combinations can further comprise one or more pharmacological or therapeutic agents or drugs such as lipid-lowering agents discussed below. As used herein, “combination therapy” or “therapeutic combination” means the administration of two or more therapeutic agents, such as a composition or therapeutic combination of the present invention (i.e., (a) an HM74 or HM74A agonist and (b) a sterol absorption inhibitor (supra)) along with a lipid-lowering or other pharmaceutical agent, to prevent or treat a condition as described above. Such administration includes coadministration of these therapeutic agents in a substantially simultaneous manner, such as in a single tablet or capsule having a fixed ratio of active ingredients or in multiple, separate capsules for each therapeutic agent. Also, such administration includes use of each type of therapeutic agent in a sequential manner. In either case, the treatment using the combination therapy will provide beneficial effects in treating the condition. A potential advantage of the combination therapy disclosed herein may be a reduction in the required amount of an individual therapeutic compound or the overall total amount of therapeutic compounds that are effective in treating the condition. By using a combination of therapeutic agents, the side effects of the individual compounds can be reduced as compared to a monotherapy, which can improve patient compliance. Also, therapeutic agents can be selected to provide a broader range of complimentary effects or complimentary modes of action.

Non-limiting examples of additional cholesterol biosynthesis inhibitors for use in the compositions, therapeutic combinations and methods of the present invention include squalene synthase inhibitors, squalene epoxidase inhibitors and mixtures thereof. Non-limiting examples of suitable HMG CoA synthetase inhibitors include lovastatin, pravastatin, fluvastatin, simvastatin, atorvastatin, rosuvastatin, cerivastatin, L-659,699 ((E, E)-11-[3′R-(hydroxy-methyl)-4′-oxo-2′R-oxetanyl]-3,5,7-R-trimethyl-2,4-undecadienoic acid); squalene synthesis inhibitors, for example squalestatin 1; and squalene epoxidase inhibitors, for example, NB-598 ((E)-N-ethyl-N-(6,6-dimethyl-2-hepten-4-ynyl)-3-[(3,3′-bithiophen-5-yl)methoxy]benzene-methanamine hydrochloride) and other sterol biosynthesis inhibitors such as DMP-565. Generally, a total daily dosage of additional cholesterol biosynthesis inhibitor(s) can range from about 0.1 to about 160 mg per day, and preferably about 0.2 to about 80 mg/day in single or 2-3 divided doses.

In another alternative embodiment, the compositions, therapeutic combinations and methods of the present invention can further comprise one or more peroxisome proliferator-activated receptor(s) (PPAR) activator(s). In this embodiment, preferably the peroxisome proliferator-activated receptor activator(s) is a fibric acid derivative such as ciprofibrate, bezafibrate, clinofibrate, binifibrate, lifibrol, gemfibrozil, clofibrate and/or fenofibrate and/or mixtures thereof.

In another alternative embodiment, the compositions, therapeutic combinations or methods of the present invention can further comprise one or more bile acid sequestrants (insoluble anion exchange resins). Bile acid sequestrants bind bile acids in the intestine, interrupting the enterohepatic circulation of bile acids and causing an increase in the faecal excretion of steroids. Bile acid sequestrants can lower intrahepatic cholesterol and promote the synthesis of apo B/E (LDL) receptors that bind LDL from plasma to further reduce cholesterol levels in the blood. Non-limiting examples of suitable bile acid sequestrants include cholestyramine (a styrenedivinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), and colesevelam hydrochloride (such as WelChol® Tablets (poly(allylamine hydrochloride) cross-linked with epichlorohydrin and alkylated with 1-bromodecane and (6-bromohexyl)-trimethylammonium bromide) which are available from Sankyo). Generally, a total daily dosage of bile acid sequestrant(s) can range from about 1 to about 50 grams per day, and preferably about 2 to about 16 grams per day in single or 2-4 divided doses.

In an alternative embodiment, the compositions, therapeutic combinations or treatments of the present invention can further comprise one or more ileal bile acid transport (“IBAT”) inhibitors (or apical sodium co-dependent bile acid transport (“ASBT”) inhibitors). The IBAT inhibitors can inhibit bile acid transport to reduce LDL cholesterol levels. Non-limiting examples of suitable IBAT inhibitors include benzothiepines such as therapeutic compounds comprising a 2,3,4,5-tetrahydro-1-benzothiepine 1,1-dioxide structure such as are disclosed in PCT Patent Application WO 00/38727 which is incorporated herein by reference. Generally, a total daily dosage of IBAT inhibitor(s) can range from about 0.01 to about 1000 mg/day, and preferably about 0.1 to about 50 mg/day in single or 2-4 divided doses.

In another alternative embodiment, the compositions, therapeutic combinations or treatments of the present invention can further comprise one or more AcylCoA:Cholesterol O-acyltransferase (“ACAT”) Inhibitors, which can reduce LDL and VLDL levels. ACAT is an enzyme responsible for esterifying excess intracellular cholesterol and may reduce the synthesis of VLDL, which is a product of cholesterol esterification, and overproduction of apo B-100-containing lipoproteins. Non-limiting examples of useful ACAT inhibitors include avasimibe, HL-004, lecimibide (DuP-128) and CL-277082 (N-(2,4-difluorophenyl)-N-[[4-(2,2-dimethylpropyl)phenyl]methyl]-N-heptylurea). See P. Chang et al., “Current, New and Future Treatments in Dyslipidaemia and Atherosclerosis”, Drugs 2000 July;60(1); 55-93, which is incorporated by reference herein. Generally, a total daily dosage of ACAT inhibitor(s) can range from about 0.1 to about 1000 mg/day in single or 2-4 divided doses.

In another alternative embodiment, the compositions, therapeutic combinations or treatments of the present invention can further comprise one or more Cholesteryl Ester Transfer Protein (“CETP”) Inhibitors. CETP is responsible for the exchange or transfer of cholesteryl ester carrying HDL and triglycerides in VLDL. Non-limiting examples of suitable CETP inhibitors are disclosed in PCT Patent Application No. WO 00/38721 and U.S. Pat. No. 6,147,090, which are incorporated herein by reference. Pancreatic cholesteryl ester hydrolase (PCEH) inhibitors such as WAY-121898 also can be coadministered with or in combination with a composition of the present invention (i.e., (a) an HM74 or HM74A, agonist and (b) a sterol absorption inhibitor) discussed above. Generally, a total daily dosage of CETP inhibitor(s) can range from about 0.01 to about 1000 mg/day, and preferably about 0.5 to about 20 mg/kg body weight/day in single or divided doses.

In another alternative embodiment, the compositions, therapeutic combinations or treatments of the present invention can further comprise probucol or derivatives thereof (such as AGI-1067 and other derivatives disclosed in U.S. Pat. Nos. 6,121,319 and 6,147,250), which can reduce LDL levels. Generally, a total daily dosage of probucol or derivatives thereof can range from about 10 to about 2000 mg/day, and preferably about 500 to about 1500 mg/day in single or 2-4 divided doses.

In another alternative embodiment, the compositions, therapeutic combinations or treatments of the present invention can further comprise low-density lipoprotein (LDL) receptor activators. Non-limiting examples of suitable LDL-receptor activators include HOE-402, an imidazolidinyl-pyrimidine derivative that directly stimulates LDL receptor activity. See M. Huettinger et al., “Hypolipidemic activity of HOE-402 is Mediated by Stimulation of the LDL Receptor Pathway”, Arterioscler. Thromb. 1993; 13:1005-12. Generally, a total daily dosage of LDL receptor activator(s) can range from about 1 to about 1000 mg/day in single or 2-4 divided doses.

In another alternative embodiment, the compositions, therapeutic combinations or treatments of the present invention can further comprise fish oil, which contains omega-3-fatty acids (3-PUFA), which can reduce VLDL and triglyceride levels. Generally, a total daily dosage of fish oil or omega 3 fatty acids can range from about 1 to about 30 grams per day in single or 2-4 divided doses.

In another alternative embodiment, the compositions, therapeutic combinations or treatments of the present invention can further comprise natural water soluble fibers, such as psyllium, guar, oat and pectin, which can reduce cholesterol levels. Generally, a total daily dosage of natural water soluble fibers can range from about 0.1 to about 10 grams per day in single or 2-4 divided doses.

In another alternative embodiment, the compositions, therapeutic combinations or treatments of the present invention can further comprise plant sterols, plant stanols and/or fatty acid esters of plant stanols, such as sitostanol ester used in BENECOL® margarine, which can reduce cholesterol levels. Generally, a total daily dosage of plant sterols, plant stanols and/or fatty acid esters of plant stanols can range from about 0.5 to about 20 grams per day in single or 2-4 divided doses.

In another alternative embodiment, the compositions, therapeutic combinations or treatments of the present invention can further comprise antioxidants, such as probucol, tocopherol, ascorbic acid, β-carotene and selenium, or vitamins such as vitamin B₆ or vitamin B₁₂. Generally, a total daily dosage of antioxidants or vitamins can range from about 0.05 to about 10 grams per day in single or 2-4 divided doses.

In another alternative embodiment, the compositions, therapeutic combinations or treatments of the present invention can further comprise monocyte and macrophage inhibitors such as polyunsaturated fatty acids (PUFA), thyroid hormones including throxine analogues such as CGS-26214 (a thyroxine compound with a fluorinated ring), gene therapy and use of recombinant proteins such as recombinant apo E. Generally, a total daily dosage of these agents can range from about 0.01 to about 1000 mg/day in single or 2-4 divided doses.

Also useful with the present invention are compositions, therapeutic combinations or treatments that further comprise hormone replacement agents and compositions. Useful hormone agents and compositions for hormone replacement therapy of the present invention include androgens, estrogens, progestins, their pharmaceutically acceptable salts and derivatives thereof. Combinations of these agents and compositions are also useful. The dosage of androgen and estrogen combinations vary, desirably from about 1 mg to about 4 mg androgen and from about 1 mg to about 3 mg estrogen.

The compositions, therapeutic combinations or methods of the present invention can further comprise one or more obesity control medications. Useful obesity control medications include, but are not limited to, drugs that reduce energy intake or suppress appetite, drugs that increase energy expenditure and nutrient-partitioning agents. Suitable obesity control medications include, but are not limited to, noradrenergic agents (such as diethylpropion, mazindol, phenylpropanolamine, phentermine, phendimetrazine, phendamine tartrate, methamphetamine, phendimetrazine and tartrate); serotonergic agents (such as sibutramine, fenfluramine, dexfenfluramine, fluoxetine, fluvoxamine and paroxtine); thermogenic agents (such as ephedrine, caffeine, theophylline, and selective β3-adrenergic agonists); alpha-blocking agents; kainite or AMPA receptor antagonists; leptin-lipolysis stimulated receptors; phosphodiesterase enzyme inhibitors; compounds having nucleotide sequences of the mahogany gene; fibroblast growth factor-10 polypeptides; monoamine oxidase inhibitors (such as befloxatone, moclobemide, brofaromine, phenoxathine, esuprone, befol, toloxatone, pirlindol, amiflamine, sercloremine, bazinaprine, lazabemide, milacemide and caroxazone); compounds for increasing lipid metabolism (such as evodiamine compounds); and lipase inhibitors (such as orlistat). Generally, a total dosage of the above-described obesity control medications can range from 1 to 3,000 mg/day, desirably from about 1 to 1,000 mg/day and more desirably from about 1 to 200 mg/day in single or 2-4 divided doses.

The compositions, therapeutic combinations or methods of the present invention can further comprise one or more blood modifiers which are chemically different from the HM74A agonists, HM74 agonists and sterol absorption inhibitors discussed above, for example, they contain one or more different atoms, have a different arrangement of atoms or a different number of one or more atoms than the HM74A agonists, HM74 agonists and sterol absorption inhibitors discussed above. Useful blood modifiers include but are not limited to anti-coagulants (argatroban, bivalirudin, dalteparin sodium, desirudin, dicumarol, lyapolate sodium, nafamostat mesylate, phenprocoumon, tinzaparin sodium, warfarin sodium); antithrombotic (anagrelide hydrochloride, bivalirudin, cilostazol, dalteparin sodium, danaparoid sodium, dazoxiben hydrochloride, efegatran sulfate, enoxaparin sodium, fluretofen, ifetroban, ifetroban sodium, lamifiban, lotrafiban hydrochloride, napsagatran, orbofiban acetate, roxifiban acetate, sibrafiban, tinzaparin sodium, trifenagrel, abciximab, zolimomab aritox); fibrinogen receptor antagonists (roxifiban acetate, fradafiban, orbofiban, lotrafiban hydrochloride, tirofiban, xemilofiban, monoclonal antibody 7E3, sibrafiban); platelet inhibitors (cilostazol, clopidogrel bisulfate, epoprostenol, epoprostenol sodium, ticlopidine hydrochloride, aspirin, ibuprofen, naproxen, sulindae, idomethacin, mefenamate, droxicam, diclofenac, sulfinpyrazone, piroxicam, dipyridamole); platelet aggregation inhibitors (acadesine, beraprost, beraprost sodium, ciprostene calcium, itazigrel, lifarizine, lotrafiban hydrochloride, orbofiban acetate, oxagrelate, fradafiban, orbofiban, tirofiban, xemilofiban); hemorrheologic agents (pentoxifylline); lipoprotein associated coagulation inhibitors; Factor VIIa inhibitors (4H-31-benzoxazin-4-ones, 4H-3,1-benzoxazin-4-thiones, quinazolin-4-ones, quinazolin-4-thiones, benzothiazin-4-ones, imidazolyl-boronic acid-derived peptide analogues TFPI-derived peptides, naphthalene-2-sulfonic acid {1-[3-(aminoiminomethyl)-benzyl]-2-oxo-pyrrolidin-3-(S)-yl}amide trifluoroacetate, dibenzofuran-2-sulfonic acid {1-[3-(aminomethyl)-benzyl]-5-oxo-pyrrolidin-3-yl}-amide, tolulene-4-sulfonic acid {1-[3-(aminoiminomethyl)-benzyl]-2-oxo-pyrrolidin-3-(S)-yl}amide trifluoroacetate, 3,4-dihydro-1H-isoquinoline-2-sulfonic acid {1-[3-(aminoiminomethyl)-benzyl]-2-oxo-pyrrolin-3-(S)-yl}-amide trifluoroacetate); Factor Xa inhibitors (disubstituted pyrazolines, disubstituted triazolines, substituted n-[(aminoiminomethyl)phenyl]propylamides, substituted n-[(aminomethyl)phenyl]propylamides, tissue factor pathway inhibitor (TFPI), low molecular weight heparins, heparinoids, benzimidazolines, benzoxazolinones, benzopiperazinones, indanones, dibasic (amidinoaryl) propanoic acid derivatives, amidinophenyl-pyrrolidines, amidinophenyl-pyrrolines, amidinophenyl-isoxazolidines, amidinoindoles, amidinoazoles, bis-arlysulfonylaminobenzamide derivatives, peptidic Factor Xa inhibitors).

The compositions, therapeutic combinations or methods of the present invention can further comprise one or more cardiovascular agents which are chemically different from the HM74A agonists, HM74 agonists and sterol absorption inhibitors discussed above, for example, they contain one or more different atoms, have a different arrangement of atoms or a different number of one or more atoms than the HM74A agonists, HM74 agonists and sterol absorption inhibitors discussed above. Useful cardiovascular agents include but are not limited to calcium channel blockers (clentiazem maleate, amlodipine besylate, isradipine, nimodipine, felodipine, nilvadipine, nifedipine, teludipine hydrochloride, diltiazem hydrochloride, belfosdil, verapamil hydrochloride, fostedil); adrenergic blockers (fenspiride hydrochloride, labetalol hydrochloride, proroxan, alfuzosin hydrochloride, acebutolol, acebutolol hydrochloride, alprenolol hydrochloride, atenolol, bunolol hydrochloride, carteolol hydrochloride, celiprolol hydrochloride, cetamolol hydrochloride, cicloprolol hydrochloride, dexpropranolol hydrochloride, diacetolol hydrochloride, dilevalol hydrochloride, esmolol hydrochloride, exaprolol hydrochloride, flestolol sulfate, labetalol hydrochloride, levobetaxolol hydrochloride, levobunolol hydrochloride, metalol hydrochloride, metoprolol, metoprolol tartrate, nadolol, pamatolol sulfate, penbutolol sulfate, practolol, propranolol hydrochloride, sotalol hydrochloride, timolol, timolol maleate, tiprenolol hydrochloride, tolamolol, bisoprolol, bisoprolol fumarate, nebivolol); adrenergic stimulants; angiotensin converting enzyme (ACE) inhibitors (benazepril hydrochloride, benazeprilat, captopril, delapril hydrochloride, fosinopril sodium, libenzapril, moexipril hydrochloride, pentopril, perindopril, quinapril hydrochloride, quinaprilat, ramipril, spirapril hydrochloride, spiraprilat, teprotide, enalapril maleate, lisinopril, zofenopril calcium, perindopril erbumine); antihypertensive agents (althiazide, benzthiazide, captopril, carvedilol, chlorothiazide sodium, clonidine hydrochloride, cyclothiazide, delapril hydrochloride, dilevalol hydrochloride, doxazosin mesylate, fosinopril sodium, guanfacine hydrochloride, methyldopa, metoprolol succinate, moexipril hydrochloride, monatepil maleate, pelanserin hydrochloride, phenoxybenzamine hydrochloride, prazosin hydrochloride, primidolol, quinapril hydrochloride, quinaprilat, ramipril, terazosin hydrochloride, candesartan, candesartan cilexetil, telmisartan, amlodipine besylate, amlodipine maleate, bevantolol hydrochloride), for example HYZAAR® or COZAAR® antihypertensive agents available from Merck & Co., Inc.; angiotensin II receptor antagonists (candesartan, irbesartan, losartan potassium, candesartan cilexetil, telmisartan); anti-anginal agents (amlodipine besylate, amlodipine maleate, betaxolol hydrochloride, bevantolol hydrochloride, butoprozine hydrochloride, carvedilol, cinepazet maleate, metoprolol succinate, molsidomine, monatepil maleate, primidolol, ranolazine hydrochoride, tosifen, verapamil hydrochloride); coronary vasodilators (fostedil, azaclorzine hydrochloride, chromonar hydrochloride, clonitrate, diltiazem hydrochloride, dipyridamole, droprenilamine, erythrityl tetranitrate, isosorbide dinitrate, isosorbide mononitrate, lidoflazine, mioflazine hydrochloride, mixidine, molsidomine, nicorandil, nifedipine, nisoldipine, nitroglycerine, oxprenolol hydrochloride, pentrinitrol, perhexiline maleate, prenylamine, propatyl nitrate, terodiline hydrochloride, tolamolol, verapamil); diuretics (the combination product of hydrochlorothiazide and spironolactone and the combination product of hydrochlorothiazide and triamterene).

The compositions, therapeutic combinations or methods of the present invention can further comprise one or more antidiabetic medications for reducing blood glucose levels in a human. Useful antidiabetic medications include, but are not limited to, drugs that reduce energy intake or suppress appetite, drugs that increase energy expenditure and nutrient-partitioning agents. Suitable antidiabetic medications include, but are not limited to, sulfonylurea (such as acetohexamide, chlorpropamide, gliamilide, gliclazide, glimepiride, glipizide, glyburide, glibenclamide, tolazamide, and tolbutamide), meglitinide (such as repaglinide and nateglinide), biguamide (such as metformin and buformin), alpha-glucosidase inhibitor (such as acarbose, miglitol, camiglibose, and voglibose), certain peptides (such as amlintide, pramlintide, exendin, and GLP-1 agonistic peptides), and orally administrable insulin or insulin composition for intestinal delivery thereof. Generally, a total dosage of the above-described antidiabetic medications can range from 0.1 to 1,000 mg/day in single or 2-4 divided doses.

The compositions, therapeutic combinations or methods of the present invention can further comprise one or more treatments for Alzheimer's Disease which are chemically different from the HM74A agonists, HM74 agonists and sterol absorption inhibitors discussed above. Non-limiting examples of suitable treatments which can be useful in treating Alzheimer's Disease include administration of one or more of the following: cholinesterase inhibitors, muscarinic receptor agonists, M2 muscarinic receptor antagonists, acetylcholine release stimulators, choline uptake stimulators, nicotinic cholinergic receptor agonists, anti-Aβ vaccines, γ-secretase inhibitors, β-secretase inhibitors, amyloid aggregation inhibitors, amyloid precursor protein antisense oligonucleotides, monoamine reuptake inhibitors, human stem cells, gene therapy, nootropic agents, AMPA receptor ligands, growth factors or growth factor receptor agonists, anti-inflammatory agents, free radical scavengers, antioxidants, superoxide dismutase stimulators, calcium channel blockers, apoptosis inhibitors, caspase inhibitors, monoamine oxidase inhibitors, estrogens and estrogen receptor ligands, NMDA receptor antagonists, Jun N-terminal kinase (JNK) inhibitors, copper/zinc chelators, 5-HT1a receptor agonists, NGF stimulators, neuroprotective agents, H₃ histamine receptor antagonists, calpain inhibitors, poly ADP ribose polymerase inhibitors, prolylendopeptidase inhibitors, calcium modulators, corticortropin releasing factor receptor antagonists, corticortropin releasing factor binding protein inhibitors, GABA modulators, GABA-A receptor antagonists, GABA-B receptor antagonists, neuroimmunophilin ligands, sigma receptor ligands, galanin receptor ligands, imidazoline/alpha adrenergic receptor antagonists, vasoactive intestinal peptide receptor agonists, benzodiazepine receptor inverse agonists, cannabinoid receptor agonists, thyrotropin releasing hormone receptor agonists, protein kinase C inhibitors, 5-HT3 receptor antagonists, prostaglandin receptor antagonists, topoisomerase 11 inhibitors, steroid receptor ligand, nitric oxide modulators, RAGE inhibitors, dopamine receptor agonists, and combinations thereof.

Mixtures of any of the pharmacological or therapeutic agents described above can be used in the compositions and therapeutic combinations of the present invention.

The compositions and therapeutic combinations of the present invention can be administered to a subject in need of such treatment in a therapeutically effective amount to treat vascular conditions such as atherosclerosis, hyperlipidaemia (including but not limited to hypercholesterolaemia, hypertriglyceridaemia, sitosterolemia), stroke, diabetes, obesity, and/or reduce the level of sterol(s) in the plasma. The compositions and treatments can be administered by any suitable means which produce contact of these compounds with the site of action in the body, for example in the plasma, liver or ileum of a mammal or human.

The daily dosage for the various compositions and therapeutic combinations described above can be administered to a patient in a single dose or in multiple subdoses, as desired. Subdoses can be administered 2 to 6 times per day, for example. Sustained release dosages can be used. Where the HM74 or HM74A agonist(s) and sterol absorption inhibitor(s) are administered in separate dosages, the number of doses of each component given per day may not necessarily be the same, e.g., one component may have a greater duration of activity and will therefore need to be administered less frequently.

Since the present invention relates to reducing the plasma sterol (especially cholesterol) concentrations or levels by treatment with a combination of active ingredients wherein the active ingredients may be administered separately, the invention also relates to combining separate pharmaceutical compositions in kit form. That is, a kit is contemplated wherein two separate units are combined: a pharmaceutical composition comprising at least one HM74 or HM74A receptor agonist and a separate pharmaceutical composition comprising at least one sterol absorption inhibitor as described above. The kit will preferably include directions for the administration of the separate components. The kit form is particularly advantageous when the separate components must be administered in different dosage forms (e.g., oral and parenteral) or are administered at different dosage intervals.

The pharmaceutical treatment compositions and therapeutic combinations of the present invention can further comprise one or more pharmaceutically acceptable carriers, one or more excipients and/or one or more additives. Non-limiting examples of pharmaceutically acceptable carriers include solids and/or liquids such as ethanol, glycerol, water and the like. The amount of carrier in the treatment composition can range from about 5 to about 99 weight percent of the total weight of the treatment composition or therapeutic combination. Non-limiting examples of suitable pharmaceutically acceptable excipients and additives include non-toxic compatible fillers, binders such as starch, disintegrants, buffers, preservatives, anti-oxidants, lubricants, flavorings, thickeners, coloring agents, emulsifiers and the like. The amount of excipient or additive can range from about 0.1 to about 90 weight percent of the total weight of the treatment composition or therapeutic combination. One skilled in the art would understand that the amount of carrier(s), excipients and additives (if present) can vary.

The treatment compositions of the present invention can be administered in any conventional dosage form, preferably an oral dosage form such as a capsule, tablet, powder, cachet, suspension or solution. The formulations and pharmaceutical compositions can be prepared using conventional pharmaceutically acceptable and conventional techniques. Several examples of preparation of dosage formulations are provided below.

The following formulations exemplify some of the dosage forms of this invention. In each formulation, the term “Active Compound I” designates a substituted azetidinone compound, β-lactam compound or any of the compounds of Formulas I-VIII described herein above and the term “Active Compound II” designates an HM74 or HM74A agonist described herein above.

### EXAMPLE

Tablets

| No. | Ingredient | mg/tablet |
|---|---|---|
| 1 | Active Compound I | 10 |
| 2 | Lactose monohydrate NF | 55 |
| 3 | Microcrystalline cellulose NF | 20 |
| 4 | Povidone (K29-32) USP | 4 |
| 5 | Croscarmellose sodium NF | 8 |
| 6 | Sodium lauryl sulfate | 2 |
| 7 | Magnesium stearate NF | 1 |
| | Total | 100 |

In the present invention, the above-described tablet can be coadministered with a tablet, capsule, etc. comprising a dosage of Active Compound II, for example a NIASPAN® niacin extended-release tablet as described above.

Method of Manufacture

Mix Item No. 4 with purified water in suitable mixer to form binder solution. Spray the binder solution and then water over Items 1, 2, 6 and a portion of Item 5 in a fluidized bed processor to granulate the ingredients. Continue fluidization to dry the damp granules. Screen the dried granules and blend with Item No. 3 and the remainder of Item 5. Add Item No. 7 and mix. Compress the mixture to appropriate size and weight on a suitable tablet machine.

For coadministration in separate tablets or capsules, representative formulations comprising a cholesterol absorption inhibitor such as are discussed above are well known in the art and representative formulations comprising an HM74 or HM74A agonist such as are discussed above are well known in the art. It is contemplated that where the two active ingredients are administered as a single composition, the dosage forms disclosed above for substituted azetidinone or β-lactam compounds may readily be modified using the knowledge of one skilled in the art.

The treatment compositions and therapeutic combinations of the present invention can inhibit the intestinal absorption of cholesterol in subjects, and can be useful in the treatment and/or prevention of vascular conditions, such as atherosclerosis, hypercholesterolemia and sitosterolemia, diabetes, obesity and lowering of plasma levels of cholesterol in subjects, in particular in mammals.

In another embodiment of the present invention, the compositions and therapeutic combinations of the present invention can inhibit sterol and/or 5α-stanol absorption or reduce plasma concentration of at least one sterol selected from the group consisting of phytosterols (such as sitosterol, campesterol, stigmasterol and avenosterol) and/or 5α-stanols (such as cholestanol, 5α-campestanol, 5α-sitostanol), cholesterol and mixtures thereof. The plasma concentration can be reduced by administering to a subject in need of such treatment an effective amount of at least one treatment composition or therapeutic combination comprising at least one HM74 or HM74A agonist and at least one sterol absorption inhibitor described above. The reduction in plasma concentration of sterols can range from about 1 to about 70 percent, and preferably about 10 to about 50 percent. Methods of measuring serum total blood cholesterol and total LDL cholesterol are well known to those skilled in the art and for example include those disclosed in PCT WO 99/38498 at page 11, incorporated by reference herein. Methods of determining levels of other sterols in serum are disclosed in H. Gylling et al., “Serum Sterols During Stanol Ester Feeding in a Mildly Hypercholesterolemic Population”, J. Lipid Res. 40: 593-600 (1999), incorporated by reference herein.

Illustrating the invention are the following examples which, however, are not to be considered as limiting the invention to their details. Unless otherwise indicated, all parts and percentages in the following examples, as well as throughout the specification, are by weight.

### Example 1

Hypercholesterolemic Golden Syrian hamsters were used to evaluate the in vivo efficacy of a cholesterol absorption inhibitor compound of Formula (XII):

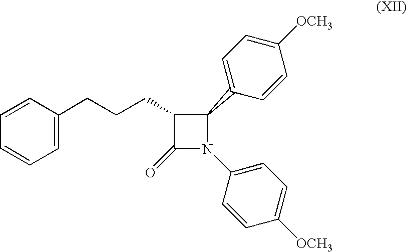

in combination with niacin. Compound XII can be prepared as shown in Example 9 of U.S. Pat. No. 5,688,787, which is incorporated by reference herein.

Hamsters were fed a cholesterol-containing diet for 7 days, which resulted in a 2-fold increase in plasma cholesterol and a 20-fold increase in hepatic cholesteryl esters. It was hypothesized that a compound which blocks dietary cholesterol absorption would reduce the accumulation of hepatic cholesteryl esters and inhibit the increase in plasma total cholesterol levels, while niacin should reduce plasma triglyceride levels.

Male Golden Syrian hamsters (Charles River Labs, Wilmington, Mass.) weighing between 100 and 125 g were fed Wayne rodent chow until study onset. At study onset (Day 1) animals were separated into groups (n=4-6/group) and fed chow supplemented with 0.5% by weight of cholesterol (Research Diets Inc., New Brunswick, N.J.). The Control and Test compounds were administered once daily for 7 days, starting on Day 1 via oral gavage in 0.2 ml corn oil. Corn oil was used as the Control. The Test compounds included the compound of Formula (XII) (3 mg/kg of body weight/day), niacin (100 mg/kg of body weight/day), or the compound of Formula (XII) combined with niacin as described in Table 1 below.

On Day 7, blood was collected into tubes containing ethylenediaminetetraacetic acid (EDTA), and plasma was prepared by low speed centrifugation at 4° C. Liver samples (0.5 g) were taken for neutral lipid analyses.

Nonfasted plasma cholesterol levels were determined by a modification of the cholesterol oxidase method, in which the reagents were available in a kit form from Wako Pure Chemicals Industries, Ltd. (Osaka, Japan). Ten μl of plasma was assayed for total cholesterol in 1 ml of 0.15 M tris buffer, pH 7.0 containing p-chlorophenol (0.1%), cholesterol oxidase (0.13 U/ml), cholesteryl ester hydrolase (0.13 U/ml), peroxidase (2.4 U/ml) and 4-aminoantipyrine (0.015%). Assays were carried out at 37° C. for 10 min, along with cholesterol standards (Sigma Chemical Co., St. Louis, Mo.) and the resultant red quinone pigment's absorbance was determined spectrophotometrically at 505 nm.

Plasma triglyceride levels were determined enzymatically by a modification of the lipase-glycerol phosphate oxidase method, obtained in a kit form from Sigma Chemical Co. Ten μl of plasma and glycerol standards were added to 1 ml of buffer, pH 7.0 containing ATP (0.3 mmol/1), Mg (3.0 mmol), 4-aminoantipyrine (0.15 mmol/1), Na N-ethyl-N-(3-sulfopropyl) n-anisidine (1.69 mmol/1), lipase (50,000 U/l), glycerol kinase (1000 U/l), glycerol phosphate oxidase (2000 U/1), and peroxidase (2000 U/l), incubated at 37° C. for 5 min., and the absorbance of the quinonemine dye was evaluated spectrophotometrically at 540 nm.

Samples of liver (0.5 g) were lipid extracted. Lipid extracts were dried under nitrogen into HPLC sample vials, resuspended in hexane and injected onto a Zorbax Sil (4.6×25 cm) silica column. Chromatography was performed using an isocratic mobile phase containing 98.8% hexane and 1.2% isopropanol at a flow rate of 2 ml/min. Lipids were detected by absorbance at 206 nm and quantitated by computer integration (System Gold, Beckman) of elution profiles. Elution time for cholesteryl ester was 1.45 min. Cholesteryl ester content of liver-derived samples was derived from a standard curve constructed using known amounts of cholesteryl oleate. Cholesteryl oleate was used as the standard since this is the major cholesteryl ester species present in the liver and this specific cholesteryl ester has an extinction coefficient that approximates that of a weighted average for all the cholesteryl esters present in the liver.

TABLE 1

| | Plasma | Plasma | Hepatic |
|---|---|---|---|
| | Cholesterol | Triglyceride | Cholesteryl Ester |
| Group | (mg/dl) | (mg/dl) | (mg/g) |
| Control | 283 ± 17 | 224 ± 21 | 24.94 ± 1.39 |
| Compound XII | 214 ± 13 | 200 ± 30 | 12.75 ± 1.12 |
| 3 mg/kg/day | | | |
| Niacin | 252 ± 17 | 169 ± 17 | 22.05 ± 1.39 |
| 100 mg/kg/day | | | |
| Compound XII | 238 ± 6 | 164 ± 28 | 14.93 ± 2.04 |
| (3 mg/kg/day) + | | | |
| Niacin | | | |
| (100 mg/kg/day) | | | |

As shown in Table 1, Compound XII reduced plasma cholesterol levels and the accumulation of hepatic cholesteryl esters in the cholesterol-fed hamsters. Niacin reduced plasma triglyceride levels, but did not significantly reduce the cholesterol levels. The combination of Compound XII and niacin resulted in reductions in plasma and hepatic cholesterol levels, as well as plasma triglycerides (Table 1). These results indicate that the combination of the cholesterol absorption inhibitor of Compound XII and niacin can have additive effects on treating hyperlipidemia in male Golden Syrian hamsters, by reducing both cholesterol and triglyceride levels.

### Example 2

### Preparation of Compound of Formula (II)

Step 1): To a solution of (S)-4-phenyl-2-oxazolidinone (41 g, 0.25 mol) in CH₂Cl₂ (200 ml), was added 4-dimethylaminopyridine (2.5 g, 0.02 mol) and triethylamine (84.7 ml, 0.61 mol) and the reaction mixture was cooled to 0° C. Methyl-4-(chloroformyl)butyrate (50 g, 0.3 mol) was added as a solution in CH₂Cl₂ (375 ml) dropwise over 1 h, and the reaction was allowed to warm to 22° C. After 17 h, water and H₂SO₄ (2N, 100 ml), was added the layers were separated, and the organic layer was washed sequentially with NaOH (10%), NaCl (sat'd) and water. The organic layer was dried over MgSO₄ and concentrated to obtain a semicrystalline product.

Step 2): To a solution of TiCl₄ (18.2 ml, 0.165 mol) in CH₂Cl₂ (600 ml) at 0° C., was added titanium isopropoxide (16.5 ml, 0.055 mol). After 15 min, the product of Step 1 (49.0 g, 0.17 mol) was added as a solution in CH₂Cl₂ (100 ml). After 5 min., diisopropylethylamine (DIPEA) (65.2 ml, 0.37 mol) was added and the reaction mixture was stirred at 0° C. for 1 h, the reaction mixture was cooled to −20° C., and 4-benzyloxybenzylidine(4-fluoro)aniline (114.3 g, 0.37 mol) was added as a solid. The reaction mixture was stirred vigorously for 4 h at −20° C., then acetic acid was added as a solution in CH₂Cl₂ dropwise over 15 min, the reaction mixture was allowed to warm to 0° C., and H₂SO₄ (2N) was added. The reaction mixture was stirred an additional 1 h, the layers were separated, washed with water, separated and the organic layer was dried. The crude product was crystallized from ethanol/water to obtain the pure intermediate.

Step 3): To a solution of the product of Step 2 (8.9 g, 14.9 mmol) in toluene (100 ml) at 500° C., was added N,O-bis(trimethylsilyl)acetamide (BSA) (7.50 ml, 30.3 mmol). After 0.5 h, solid TBAF (0.39 g, 1.5 mmol) was added and the reaction mixture stirred at 50° C. for an additional 3 h. The reaction mixture was cooled to 22° C., CH₃OH (10 ml), was added. The reaction mixture was washed with HCl (1N), NaHCO₃ (1N) and NaCl (sat'd.), and the organic layer was dried over MgSO₄.

Step 4): To a solution of the product of Step 3 (0.94 g, 2.2 mmol) in CH₃OH (3 ml), was added water (1 ml) and LiOH.H₂O (102 mg, 2.4 mmole). The reaction mixture was stirred at 22° C. for 1 h and then additional LiOH.H₂O (54 mg, 1.3 mmole) was added. After a total of 2 h, HCl (1N) and EtOAc was added, the layers were separated, the organic layer was dried and concentrated in vacuo. To a solution of the resultant product (0.91 g, 2.2 mmol) in CH₂Cl₂ at 22° C., was added ClCOCOCl (0.29 ml, 3.3 mmol) and the mixture stirred for 16 h. The solvent was removed in vacuo.

Step 5): To an efficiently stirred suspension of 4-fluorophenylzinc chloride (4.4 mmol) prepared from 4-fluorophenylmagnesium bromide (1M in THF, 4.4 ml, 4.4 mmol) and ZnCl₂ (0.6 g, 4.4 mmol) at 40° C., was added tetrakis(triphenylphosphine)palladium (0.25 g, 0.21 mmol) followed by the product of Step 4 (0.94 g, 2.2 mmol) as a solution in THF (2 ml). The reaction was stirred for 1 h at 0° C. and then for 0.5 h at 22° C. HCl (1N, 5 ml) was added and the mixture was extracted with EtOAc. The organic layer was concentrated to an oil and purified by silica gel chromatography to obtain 1-(4-fluorophenyl)-4(S)-(4-hydroxyphenyl)-3(R)-(3-oxo-3-phenylpropyl)-2-azetidinone:

HRMS calc'd for C₂₄H₁₉F₂NO₃=408.1429, found 408.1411.

Step 6): To the product of Step 5 (0.95 g, 1.91 mmol) in THF (3 ml), was added (R)-tetrahydro-1-methyl-3,3-diphenyl-1H,3H-pyrrolo-[1,2-c][1,3,2] oxazaborole (120 mg, 0.43 mmol) and the mixture was cooled to −20° C. After 5 min, borohydride-dimethylsulfide complex (2M in THF, 0.85 ml, 1.7 mmol) was added dropwise over 0.5 h. After a total of 1.5 h, CH₃OH was added followed by HCl (1N) and the reaction mixture was extracted with EtOAc to obtain 1-(4-fluorophenyl)-3(R)-[3(S)-(4-fluorophenyl)-3-hydroxypropyl)]-4(S)-[4-(phenylmethoxy)phenyl]-2-azetidinone (compound 6A-1) as an oil. ¹H in CDCl₃ d H₃=4.68. J=2.3 Hz. Cl (M+H) 500.

Use of (S)-tetra-hydro-1-methyl-3,3-diphenyl-1H,3H-pyrrolo-[1,2-c][1,3,2]oxazaborole gives the corresponding 3(R)-hydroxypropyl azetidinone (compound 6B-1). ¹H in CDCl₃ d H₃=4.69. J=2.3 Hz. Cl (M⁺H) 500.

To a solution of compound 6A-1 (0.4 g, 0.8 mmol) in ethanol (2 ml), was added 10% Pd/C (0.03 g) and the reaction mixture was stirred under a pressure (60 psi) of H₂ gas for 16 h. The reaction mixture was filtered and the solvent was concentrated to obtain compound 6A. Mp 164-166° C.; Cl (M+H) 410. [α]_{D}²⁵=28.1° (c 3, CH₃OH) Elemental analysis calc'd for C₂₄H₂₁F₂NO₃: C 70.41; H 5.17; N 3.42; found C 70.25; H 5.19; N 3.54.

Similarly treat compound 6B-1 to obtain compound 6B.

Mp 129.5-132.5° C.; Cl (M⁺H) 410. Elemental analysis calc'd for C₂₄H₂₁F₂NO₃: C 70.41; H 5.17; N 3.42; found C 70.30; H 5.14; N 3.52.

Step 6′ (Alternative): To a solution of the product of Step 5 (0.14 g, 0.3 mmol) in ethanol (2 ml), was added 10% Pd/C (0.03 g) and the reaction was stirred under a pressure (60 psi) of H₂ gas for 16 h. The reaction mixture was filtered and the solvent was concentrated to afford a 1:1 mixture of compounds 6A and 6B.

It will be appreciated by those skilled in the art that changes could be made to the embodiments described above without departing from the broad inventive concept thereof. It is understood, therefore, that this invention is not limited to the particular embodiments disclosed, but it is intended to cover modifications which are within the spirit and scope of the invention, as defined by the appended claims.

Patents, patent applications, publications, product descriptions, and protocols are cited throughout this application, the disclosures of which are incorporated herein by reference in their entireties for all purposes.

Therefore, we claim:

1. A composition comprising:
(c) at least one HM74 or HM74A agonist; and
(d) at least one sterol absorption inhibitor selected from the group consisting of:
(i) a compound represented by Formula (I): 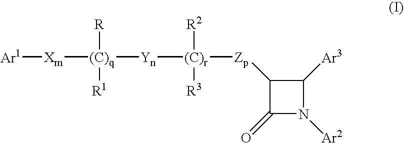
or pharmaceutically acceptable salts or solvates thereof,
wherein in Formula (I) above:
Ar¹ and Ar² are independently selected from the group consisting of aryl and R⁴-substituted aryl;
Ar³ is aryl or R⁵-substituted aryl;
X, Y and Z are independently selected from the group consisting of —CH₂—, —CH(lower alkyl)- and —C(dilower alkyl)-;
R and R² are independently selected from the group consisting of —OR⁶, —O(CO)R⁶, —O(CO)OR⁹ and —O(CO)NR⁶R⁷;
R¹ and R³ are independently selected from the group consisting of hydrogen, lower alkyl and aryl;
q is 0 or 1;
r is 0 or 1;
m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶, —CH═CH—COOR⁶, CF₃, —CN, —NO₂ and halogen;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl;
(ii) a compound represented by Formula (III): 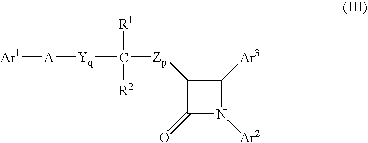
or pharmaceutically acceptable salts or solvates thereof wherein, in Formula (III) above:
Ar¹ is R³-substituted aryl;
Ar² is R⁴-substituted aryl;
Ar³ is R⁵-substituted aryl;
Y and Z are independently selected from the group consisting of —CH₂—, —CH(lower alkyl)- and —C(dilower alkyl)-;
A is selected from —O—, —S—, —S(O)— or —S(O)₂—;
R¹ is selected from the group consisting of —OR⁶, —O(CO)R⁶, —O(CO)OR⁹ and —O(CO)NR⁶R⁷; R² is selected from the group consisting of hydrogen, lower alkyl and aryl; or R¹ and R² together are ═O;
q is 1, 2 or 3;
p is 0, 1, 2, 3 or 4;
R⁵ is 1-3 substituents independently selected from the group consisting of —OR⁶, —O(CO)R⁶, —O(CO)OR⁹, —O(CH₂)₁₋₅OR⁹, —O(CO)NR⁶R⁷, —NR⁶R⁷, —NR⁶(CO)R⁷, —NR⁶(CO)OR⁹, —NR⁶(CO)NR⁶R⁷, —NR⁶SO₂-lower alkyl, —NR⁶SO₂-aryl, —CONR⁶R⁷, —COR⁶, —SO₂NR⁶R⁷, S(O)₀₋₂-alkyl, S(O)₀₋₂-aryl, —O(CH₂)₁₋₁₀—COOR⁶, —O(CH₂)₁₋₁₀CONR⁶R⁷, o-halogeno, m-halogeno, o-lower alkyl, m-lower alkyl, -(lower alkylene)-COOR⁶, and —CH═CH—COOR⁶;
R³ and R⁴ are independently 1-3 substituents independently selected from the group consisting of R⁵, hydrogen, p-lower alkyl, aryl, —NO₂, —CF₃ and p-halogeno;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl;
(iii) a compound represented by Formula (IV): 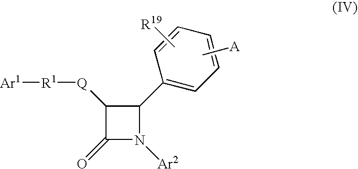
or pharmaceutically acceptable salts or solvates wherein, in Formula (IV) above:
A is selected from the group consisting of R²-substituted heterocycloalkyl, R²-substituted heteroaryl, R²-substituted benzofused heterocycloalkyl, and R²-substituted benzofused heteroaryl;
Ar¹ is aryl or R³-substituted aryl;
Ar¹ is aryl or R⁴-substituted aryl;
Q is a bond or, with the 3-position ring carbon of the azetidinone, forms the spiro group 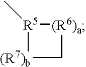
and
R¹ is selected from the group consisting of:
—(CH₂)_{q}—, wherein q is 2-6, provided that when Q forms a spiro ring, q can also be zero or 1;
—(CH₂)ₑ-G-(CH₂)ᵣ—, wherein G is —O—, —C(O)—, phenylene, —NR⁸— or —S(O)₀₋₂—, e is 0-5 and r is 0-5, provided that the sum of e and r is 1-6;
—(C₂-C₆ alkenylene)-; and
—(CH₂)_{f}-V-(CH₂)_{g}—, wherein V is C₃-C₆ cycloalkylene, f is 1-5 and g is 0-5, provided that the sum of f and g is 1-6;
R⁵ is selected from: 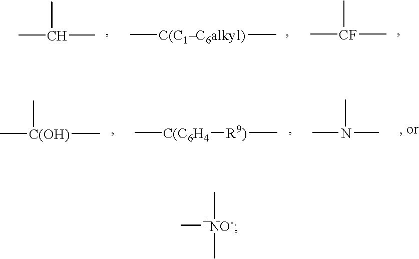
R⁶ and R⁷ are independently selected from the group consisting of —CH₂—, —CH(C₁-C₆ alkyl)-, —C(di-(C₁-C₆) alkyl), —CH═CH— and —C(C₁-C₆ alkyl)═CH—; or R⁵ together with an adjacent R⁶, or R⁵ together with an adjacent R⁷, form a —CH═CH— or a —CH═C(C₁-C₆ alkyl)- group;
a and b are independently 0, 1, 2 or 3, provided both are not zero; provided that when R⁶ is —CH═CH— or —C(C₁-C₆ alkyl)═CH—, a is 1; provided that when R⁷ is —CH═CH— or —C(C₁-C₆ alkyl)═CH—, b is 1; provided that when a is 2 or 3, the R⁶'s can be the same or different; and provided that when b is 2 or 3, the R⁷'s can be the same or different;
and when Q is a bond, R¹ also can be selected from: 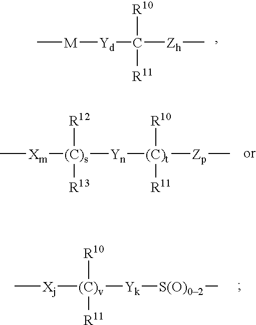
where M is —O—, —S—, —S(O)— or —S(O)₂—;
X, Y and Z are independently selected from the group consisting of —CH₂—, —CH(C₁-C₆ alkyl)- and —C(di-(C₁-C₆) alkyl);
R¹⁰ and R¹² are independently selected from the group consisting of —OR¹⁴, —O(CO)R¹⁴, —O(CO)OR¹⁶ and —O(CO)NR¹⁴R¹⁵;
R¹¹ and R¹³ are independently selected from the group consisting of hydrogen, (C₁-C₆)alkyl and aryl; or R¹⁰ and R¹¹ together are ═O, or R¹² and R¹³ together are ═O;
d is 1, 2 or 3;
h is 0, 1, 2, 3 or 4;
s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;
v is 0 or 1;
j and k are independently 1-5, provided that the sum of j, k and v is 1-5;
R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-, —NHC(O)R¹⁶, OH, C₁-C₆ alkoxy, —OC(O)R¹⁶, —COR¹⁴, hydroxy(C₁-C₆)alkyl, (C₁-C₆)alkoxy(C₁-C₆)alkyl, NO₂, —S(O)₀₋₂R¹⁶, —SO₂NR¹⁴ R¹⁵ and —(C₁-C₆ alkylene)COOR¹⁴; when R² is a substituent on a heterocycloalkyl ring, R² is as defined, or is ═O or 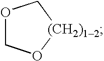
and, where R² is a substituent on a substitutable ring nitrogen, it is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸, 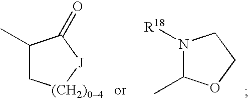
wherein J is —O—, —NH—, —NR¹⁸— or —CH₂—;
R³ and R⁴ are independently selected from the group consisting of 1-3 substituents independently selected from the group consisting of (C₁-C₆)alkyl, —OR¹⁴, —O(CO)R¹⁴, —O(CO)OR¹⁶, —O(CH₂)₁₋₅OR¹⁴, —O(CO)NR¹⁴R¹⁵, —NR¹⁴R¹⁵, —NR¹⁴(CO)R¹⁵, —NR¹⁴(CO)OR¹⁶, NR¹⁴(CO)NR¹⁵R¹⁹, —NR¹⁴SO₂R¹⁶, —COOR¹⁴, —CONR¹⁴R¹⁵, —COR¹⁴, —SO₂NR¹⁴R¹⁵, S(O)₀₋₂R¹⁶, —O(CH₂)₁₋₁₀—COOR¹⁴, —O(CH₂)₁₋₁₀CONR¹⁴R¹⁵, —(C₁-C₆ alkylene)-COOR¹⁴, —CH═CH—COOR¹⁴, —CF₃, —CN, —NO₂ and halogen;
R⁸ is hydrogen, (C₁-C₆)alkyl, aryl (C₁-C₆)alkyl, —C(O)R¹⁴ or —COOR¹⁴;
R⁹ and R¹⁷ are independently 1-3 groups independently selected from the group consisting of hydrogen, (C₁-C₆)alkyl, (C₁-C₆)alkoxy, —COOH, NO_{21 , —NR}¹⁴R¹⁵, OH and halogeno;
R¹⁴ and R¹⁵ are independently selected from the group consisting of hydrogen, (C₁-C₆)alkyl, aryl and aryl-substituted (C₁-C₆)alkyl;
R¹⁶ is (C₁-C₆)alkyl, aryl or R¹⁷-substituted aryl;
R¹⁸ is hydrogen or (C₁-C₆)alkyl; and
R¹⁹ is hydrogen, hydroxy or (C₁-C₆)alkoxy;
(iv) a compound represented by Formula (V): 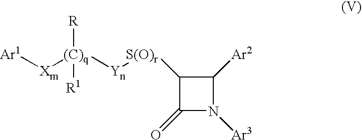
or pharmaceutically acceptable salts or solvates thereof, wherein, in Formula (V) above:
Ar¹ is aryl, R¹⁰-substituted aryl or heteroaryl;
Ar² is aryl or R₄-substituted aryl;
Ar³ is aryl or R⁵-substituted aryl;
X and Y are independently selected from the group consisting of —CH₂—, —CH(lower alkyl)- and —C(dilower alkyl)-;
R is —OR⁶, —O(CO)R⁶, —O(CO)OR⁹ or —O(CO)NR⁶R⁷; R¹ is hydrogen, lower alkyl or aryl; or R and R¹ together are ═O;
q is 0 or 1;
r is 0, 1 or 2;
m and n are independently 0, 1, 2, 3, 4 or 5; provided that the sum of m, n and q is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂, halogen, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl;
R⁹ is lower alkyl, aryl or aryl-substituted lower alkyl; and
R¹⁰ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O(CH₂)₁₋₁₀—COOR⁶, —O(CH₂)₁₋₁₀CONR⁶R⁷, —CF₃, —CN, —NO₂ and halogen;
(v) a compound represented by Formula (VI): 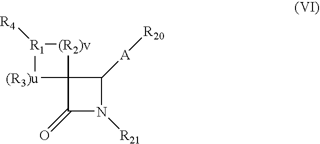
or pharmaceutically acceptable salts or solvates thereof, wherein:
R₁ is 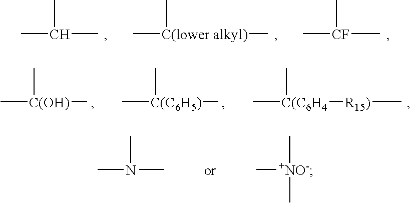
R₂ and R₃ are independently selected from the group consisting of: —CH₂—, —CH(lower alkyl)-, —C(di-lower alkyl)-, —CH═CH— and —C(lower alkyl)═CH—; or R₁ together with an adjacent R₂, or R₁ together with an adjacent R₃, form a —CH═CH— or a —CH═C(lower alkyl)- group;
u and v are independently 0, 1, 2 or 3, provided both are not zero; provided that when R₂ is —CH═CH— or —C(lower alkyl)═CH—, v is 1; provided that when R₃ is —CH═CH— or —C(lower alkyl)═CH—, u is 1; provided that when v is 2 or 3, the R₂'s can be the same or different; and provided that when u is 2 or 3, the R₃'s can be the same or different;
R₄ is selected from B—(CH₂)ₘC(O)—, wherein m is 0, 1, 2, 3, 4 or 5;
B—(CH₂)_{q}—, wherein q is 0, 1, 2, 3, 4, 5 or 6;
B—(CH₂)ₑ-Z-(CH₂)ᵣ—, wherein Z is —O—, —C(O)—, phenylene, —N(R₈)— or —S(O)₀₋₂-, e is 0, 1, 2, 3, 4 or 5 and r is 0, 1, 2, 3, 4 or 5, provided that the sum of e and r is 0, 1, 2, 3, 4, 5 or 6;
B—(C₂-C₆ alkenylene)-;
B—(C₄-C₆ alkadienylene)-;
B—(CH₂)ₜ-Z-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6;
B—(CH₂)_{f}-V-(CH₂)_{g}—, wherein V is C₃-C₆ cycloalkylene, f is 1, 2, 3, 4 or 5 and g is 0, 1, 2, 3, 4 or 5, provided that the sum of f and g is 1, 2, 3, 4, 5 or 6;
B—(CH₂)ₜ-V-(C₂-C₆ alkenylene)- or
B—(C₂-C₆ alkenylene)-V-(CH₂)ₜ—, wherein V and t are as defined above, provided that the sum of t and the number of carbon atoms in the alkenylene chain is 2, 3, 4, 5 or 6;
B—(CH₂)ₐ-Z-(CH₂)_{b}-V-(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or
T-(CH₂)ₛ—, wherein T is cycloalkyl of 3-6 carbon atoms and s is 0, 1, 2, 3, 4, 5 or 6; or
R₁ and R₄ together form the group 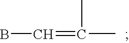
B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or 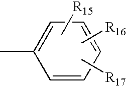
W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R₇-benzyl, benzyloxy, R₇-benzyloxy, phenoxy, R₇-phenoxy, dioxolanyl, NO₂, —N(R₈)(R₉), N(R₈)(R₉)-lower alkylene-, N(R₈)(R₉)-lower alkylenyloxy-, OH, halogeno, —CN, —N₃, —NHC(O)OR₁₀, —NHC(O)R₁₀, R₁₁O₂SNH—, (R₁₁O₂S)₂N—, —S(O)₂NH₂, —S(O)₀₋₂R₈, tert-butyldimethyl-silyloxymethyl, —C(O)R₁₂, —COOR₁₉, —CON(R₈)(R₉), —CH═CHC(O)R₁₂, -lower alkylene-C(O)R₁₂, R₁₀C(O)(lower alkylenyloxy)-, N(R₈)(R₉)C(O)(lower alkylenyloxy)- and 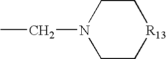
for substitution on ring carbon atoms, and the substituents on the substituted heteroaryl ring nitrogen atoms, when present, are selected from the group consisting of lower alkyl, lower alkoxy, C(O)OR₁₀, —C(O)R₁₀, OH, N(R₈)(R₉)-lower alkylene-, N(R₈)(R₉)-lower alkylenyloxy-, —S(O)₂NH₂ and 2-(trimethylsilyl)-ethoxymethyl;
R₇ is 1-3 groups independently selected from the group consisting of lower alkyl, lower alkoxy, —COOH, NO₂, —N(R₈)(R₉), OH, and halogeno;
R₈ and R₉ are independently selected from H or lower alkyl;
R₁₀ is selected from lower alkyl, phenyl, R₇-phenyl, benzyl or R₇-benzyl;
R₁₁ is selected from OH, lower alkyl, phenyl, benzyl, R₇-phenyl or R₇-benzyl;
R₁₂ is selected from H, OH, alkoxy, phenoxy, benzyloxy, 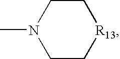
—N(R₈)(R₉), lower alkyl, phenyl or R₇-phenyl;
R₁₃ is selected from —O—, —CH₂—, —NH—, —N(lower alkyl)- or —NC(O)R₁₉;
R₁₅, R₁₆ and R₁₇ are independently selected from the group consisting of H and the groups defined for W; or R₁₅ is hydrogen and R₁₆ and R₁₇, together with adjacent carbon atoms to which they are attached, form a dioxolanyl ring;
R₁₉ is H, lower alkyl, phenyl or phenyl lower alkyl; and
R₂₀ and R₂₁ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzofused heteroaryl, W-substituted benzofused heteroaryl and cyclopropyl, wherein heteroaryl is as defined above;
(vi) a compound represented by Formula (VIIA) or (VIIB): 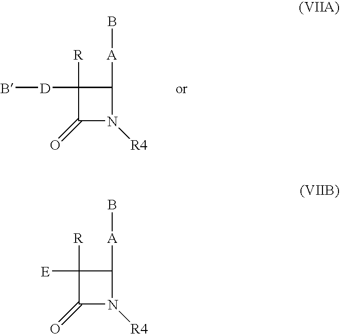
or pharmaceutically acceptable salts or solvates thereof, wherein in Formulae (VIIA) and (VIIB):
A is —CH═CH—, —C≡C— or —(CH₂)ₚ— wherein p is 0, 1 or 2;
B is 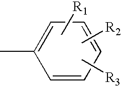
B′ is 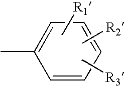
D is —(CH₂)ₘC(O)— or —(CH₂)_{q}— wherein m is 1, 2, 3 or 4 and q is 2, 3 or 4;
E is C_{10 to C}₂₀ alkyl or —C(O)—(₉ to C₁₉)-alkyl, wherein the alkyl is straight or branched, saturated or containing one or more double bonds;
R is hydrogen, C₁-C₁₅ alkyl, straight or branched, saturated or containing one or more double bonds, or B—(CH₂)ᵣ-, wherein r is 0, 1, 2, or 3;
R₁, R₂, R₃, R_{1′}, R_{2′}, and R_{3′} are independently selected from the group consisting of hydrogen, lower alkyl, lower alkoxy, carboxy, NO₂, NH₂, OH, halogeno, lower alkylamino, dilower alkylamino, —NHC(O)OR₅, R₆O₂SNH— and —S(O)₂NH₂;
R₄ is 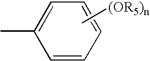
wherein n is 0, 1, 2 or 3;
R₅ is lower alkyl; and
R₆ is OH, lower alkyl, phenyl, benzyl or substituted phenyl wherein the substituents are 1-3 groups independently selected from the group consisting of lower alkyl, lower alkoxy, carboxy, NO₂, NH₂, OH, halogeno, lower alkylamino and dilower alkylamino;
(vii) a compound represented by Formula (VIII): 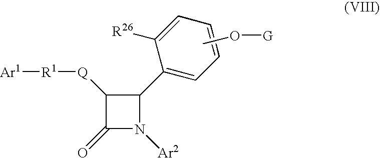
or pharmaceutically acceptable salts or solvates thereof, wherein, in Formula (VIII) above,
R²⁶ is H or OG¹;
G and G¹ are independently selected from the group consisting of 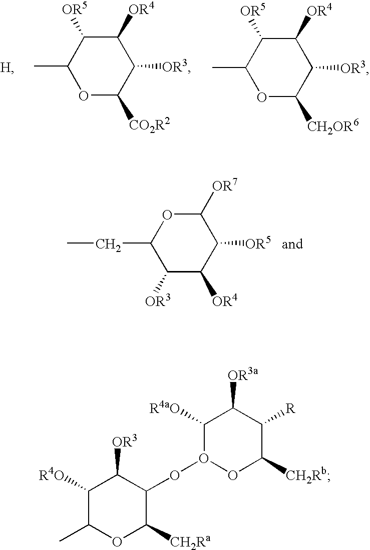
provided that when R²⁶ is H or OH, G is not H;
R, R^{a} and R^{b} are independently selected from the group consisting of H, —OH, halogeno, —NH₂, azido, (C₁-C₆)alkoxy(C₁-C₆)-alkoxy or —W—R³⁰;
W is independently selected from the group consisting of —NH—C(O)—, —O—C(O)—, —O—C(O)—N(R³¹)—, —NH—C(O)—N(R³¹)— and —O—C(S)—N(R³¹)—;
R² and R⁶ are independently selected from the group consisting of H, (C₁-C₆)alkyl, aryl and aryl(C₁-C₆)alkyl;
R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;
R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;
R³¹ is selected from the group consisting of H and (C₁-C₄)alkyl;
T is selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;
R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;
Ar¹ is aryl or R¹⁰-substituted aryl;
Ar² is aryl or R¹¹-substituted aryl;
Q is a bond or, with the 3-position ring carbon of the azetidinone, forms the spiro group 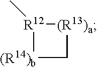
and
R¹ is selected from the group consisting of
—(CH₂)_{q}—, wherein q is 2-6, provided that when Q forms a spiro ring, q can also be zero or 1;
—(CH₂)ₑ-E-(CH₂)ᵣ—, wherein E is —O—, —C(O)—, phenylene, —NR²²— or —S(O)₀₋₂-, e is 0-5 and r is 0-5, provided that the sum of e and r is 1-6;
—(C₂-C₆)alkenylene-; and
—(CH₂)_{f}-V-(CH₂)_{g}—, wherein V is C₃-C₆ cycloalkylene, f is 1-5 and g is 0-5, provided that the sum of f and g is 1-6;
R¹² is 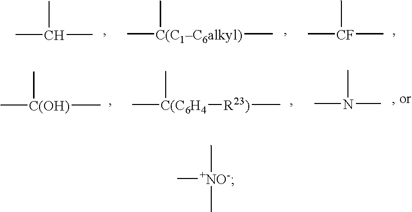
R¹³ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)═CH—; or R¹² together with an adjacent R¹³, or R¹² together with an adjacent R¹⁴, form a —CH═CH— or a —CH═C(C₁-C₆ alkyl)- group;
a and b are independently 0, 1, 2 or 3, provided both are not zero;
provided that when R¹³ is —CH═CH— or —C(C₁-C₆ alkyl)═CH—, a is 1;
provided that when R¹⁴ is —CH═CH— or —C(C₁-C₆ alkyl)═CH—, b is 1;
provided that when a is 2 or 3, the R¹³'s can be the same or different; and
provided that when b is 2 or 3, the R¹⁴'s can be the same or different;
and when Q is a bond, R¹ also can be: 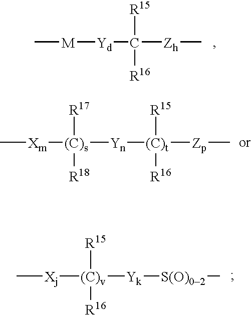
M is —O—, —S—, —S(O)— or —S(O)₂—;
X, Y and Z are independently selected from the group consisting of —CH₂—, —CH(C₁-C₆)alkyl- and —C(di-(C₁-C₆)alkyl);
R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR² R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;
R¹⁵ and R¹⁷ are independently selected from the group consisting of —OR¹⁹, —O(CO)R¹⁹, —O(CO)OR²¹ and —O(CO)NR¹⁹R²⁰;
R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;
d is 1, 2 or 3;
h is 0, 1, 2, 3 or 4;
s is 0 or 1; t is 0 or 1; m, n and p are independently 0-4;
provided that at least one of s and t is 1, and the sum of m, n, p, s and t is 1-6;
provided that when p is 0 and t is 1, the sum of m, s and n is 1-5; and provided that when p is 0 and s is 1, the sum of m, t and n is 1-5;
v is 0 or 1;
j and k are independently 1-5, provided that the sum of j, k and v is 1-5;
and when Q is a bond and R¹ is 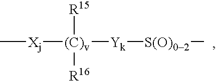
Ar¹ can also be pyridyl, isoxazolyl, furanyl, pyrrolyl, thienyl, imidazolyl, pyrazolyl, thiazolyl, pyrazinyl, pyrimidinyl or pyridazinyl;
R¹⁹ and R²⁰ are independently selected from the group consisting of H, (C₁-C₆)alkyl, aryl and aryl-substituted (C₁-C₆)alkyl;
R²¹ is (C₁-C₆)alkyl, aryl or R²⁴-substituted aryl;
R²² is H, (C₁-C₆)alkyl, aryl (C₁-C₆)alkyl, —C(O)R¹⁹ or —COOR¹⁹;
R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and
R²⁵ is H, —OH or (C₁-C₆)alkoxy; and
(viii) a compound represented by Formula (IX): 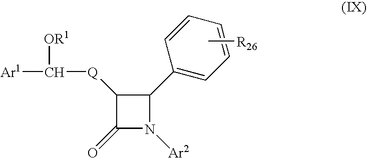
or pharmaceutically acceptable salts or solvates thereof, wherein in Formula (IX):
R²⁶ is selected from the group consisting of:
a) OH;
b) OCH₃;
c) fluorine and
d) chlorine.
R¹ is selected from the group consisting of 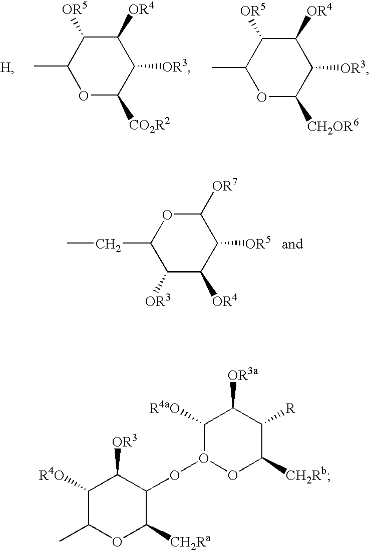
—SO₃H; natural and unnatural amino acids.
R, R^{a} and R^{b} are independently selected from the group consisting of H, —OH, halogeno, —NH₂, azido, (C₁-C₆)alkoxy(C₁-C₆)-alkoxy and —W—R³⁰;
W is independently selected from the group consisting of —NH—C(O)—, —O—C(O)—, —O—C(O)—N(R³¹)—, —NH—C(O)—N(R³¹)— and —O—C(S)—N(R³¹)—;
R² and R⁶ are independently selected from the group consisting of H, (C₁-C₆)alkyl, aryl and aryl(C₁-C₆)alkyl;
R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;
R³⁰ is independently selected form the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;
R³¹ is independently selected from the group consisting of H and (C₁-C₄)alkyl;
T is independently selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;
R³² is independently selected from 1-3 substituents independently selected from the group consisting of H, halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;
Ar¹ is aryl or R¹⁰-substituted aryl;
Ar² is aryl or R¹¹-substituted aryl;
Q is —(CH₂)_{q}—, wherein q is 2-6, or, with the 3-position ring carbon of the azetidinone,
forms the spiro group 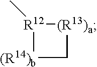
R¹² is 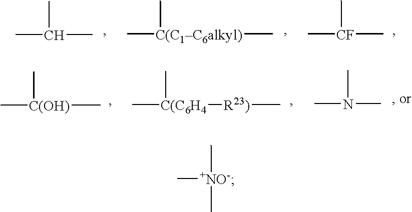
R¹³ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)═CH—; or R¹² together with an adjacent R¹³, or R¹² together with an adjacent R¹⁴, form a —CH═CH— or a —CH═C(C₁-C₆ alkyl)- group;
a and b are independently 0, 1, 2 or 3, provided both are not zero; provided that when R¹³ is —CH═CH— or —C(C₁-C₆ alkyl)═CH—, a is 1; provided that when R¹⁴ is —CH═CH— or —C(C₁-C₆ alkyl)═CH—, b is 1; provided that when a is 2 or 3, the R¹³'s can be the same or different; and provided that when b is 2 or 3, the R¹⁴'s can be the same or different;
R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;
Ar¹ can also be pyridyl, isoxazolyl, furanyl, pyrrolyl, thienyl, imidazolyl, pyrazolyl, thiazolyl, pyrazinyl, pyrimidinyl or pyridazinyl;
R¹⁹ and R²⁰ are independently selected from the group consisting of H, (C₁-C₆)alkyl, aryl and aryl-substituted (C₁-C₆)alkyl;
R²¹ is (C₁-C₆)alkyl, aryl or R²⁴-substituted aryl;
R²² is H, (C₁-C₆)alkyl, aryl (C₁-C₆)alkyl, —C(O)R¹⁹ or —COOR¹⁹;
R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and
R²⁵ is H, —OH or (C₁-C₆)alkoxy.

2. The composition according to claim 1, wherein the HM74 or HM74A agonist is nicotinic acid or a nicotinic acid derivative.

3. The composition of claim 2, wherein the HM74 or HM74A agonist is a nicotinic acid derivative selected from the group consisting of pyradine-3-acetic acid, 5-methyl nicotinic acid, nicotinuric acid, niceritrol, nicofuranose, 5-methylpyrazine-2-carboxylic acid 4-oxide and any pharmaceutically acceptable salt or solvate thereof.

4. The composition of claim 1, wherein the HM74 or HM74A agonist is 5-methyl pyrazole-3-carboxylic acid or acifran.

5. The composition according to claim 1, wherein the at least one of HM74 or HM74A agonist is administered to a subject in an amount ranging from about 500 to about 10,000 milligrams per day.

6. The composition according to claim 1, wherein the sterol absorption inhibitor is represented by Formula (II) below:

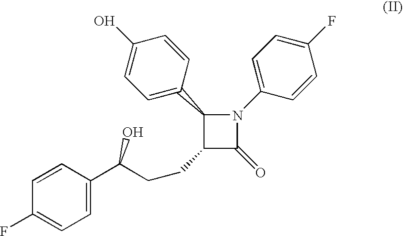

7. The composition according to claim 1, wherein the at least one sterol absorption inhibitor is administered to a subject in an amount ranging from about 0.1 to about 1000 milligrams of sterol absorption inhibitor per day.

8. The composition according to claim 1, further comprising at least one cholesterol biosynthesis inhibitor.

9. The composition according to claim 8, wherein the cholesterol biosynthesis inhibitor comprises at least one HMG CoA reductase inhibitor.

10. The composition according to claim 9, wherein the HMG CoA reductase inhibitor is selected from the group consisting of lovastatin, pravastatin, fluvastatin, simvastatin, atorvastatin, rosuvastatin, cerivastatin and mixtures thereof.

11. The composition according to claim 10, wherein the HMG CoA reductase inhibitor is simvastatin.

12. The composition according to claim 1, further comprising a lipid lowering agent selected from the group consisting of a peroxisome proliferator-activated receptor (PPAR) activator, a bile acid sequestrant, an AcylCoA:Cholesterol O-acyltransferase Inhibitor, probucol, a derivative of probucol, a low-density lipoprotein receptor activator, an omega-3-fatty acid, a natural water soluble fiber, a plant sterol, a plant stanol and a fatty acid ester of a plant stanol.

13. The composition according to claim 1, further comprising at least one additive selected from the group consisting of an antioxidant, a vitamin, a hormone replacement therapy composition, an obesity control medication, a blood modifier, a cardiovascular agent different from the compounds of Formulae I-IX and an antidiabetic medication.

14. A pharmaceutical composition for the treatment or prevention of a vascular condition, diabetes, obesity or lowering a concentration of a sterol in plasma of a subject, comprising a therapeutically effective amount of the composition of claim 1 and a pharmaceutically acceptable carrier.

15. A method of treating or preventing a vascular condition, diabetes, obesity or lowering a concentration of a sterol in plasma of a subject, comprising the step of administering to a subject in need of such treatment an effective amount of the composition of claim 1.

16. The method according to claim 15, wherein the vascular condition is hyperlipidemia.

17. A therapeutic combination comprising:
(a) a first amount of at least one HM74 or HM74A agonist; and
(b) a second amount of at least one sterol absorption inhibitor or a pharmaceutically acceptable salt or solvate thereof,
wherein the first amount and the second amount together comprise a therapeutically effective amount for the treatment or prevention of a vascular condition, diabetes, obesity or lowering a concentration of a sterol in plasma of a subject.

18. A pharmaceutical composition for the treatment or prevention of a vascular condition, diabetes, obesity or lowering a concentration of a sterol in plasma of a subject, comprising a therapeutically effective amount of the therapeutic combination of claim 17 and a pharmaceutically acceptable carrier.

19. A method of treating or preventing a vascular condition, diabetes, obesity or lowering a concentration of a sterol in plasma of a subject, comprising the step of administering to a subject in need of such treatment an effective amount of the composition of claim 17.

20. A composition comprising: (a) at least one HM74 or HM74A agonist; and
(b) at least one substituted azetidinone compound or a pharmaceutically acceptable salt or solvate thereof.

21. A pharmaceutical composition for the treatment or prevention of a vascular condition, diabetes, obesity or lowering a concentration of a sterol in plasma of a subject, comprising a therapeutically effective amount of the composition of claim 20 and a pharmaceutically acceptable carrier.

22. A method of treating or preventing a vascular condition, diabetes, obesity or lowering a concentration of a sterol in plasma of a subject, comprising the step of administering to a subject in need of such treatment an effective amount of the composition of claim 20.

23. A therapeutic combination comprising:
(a) a first amount of at least one HM74 or HM74A agonist; and
(c) a second amount of at least one substituted azetidinone compound or a substituted β-lactam compound or a pharmaceutically acceptable salt or solvate thereof,
wherein the first amount and the second amount together comprise a therapeutically effective amount for the treatment or prevention of a vascular condition, diabetes, obesity or lowering a concentration of a sterol in plasma of a subject.

24. A pharmaceutical composition for the treatment or prevention of a vascular condition, diabetes, obesity or lowering a concentration of a sterol in plasma of a subject, comprising a therapeutically effective amount of the therapeutic combination of claim 23 and a pharmaceutically acceptable carrier.

25. A method of treating or preventing a vascular condition, diabetes, obesity or lowering a concentration of a sterol in plasma of a subject, comprising the step of administering to a subject in need of such treatment an effective amount of the composition of claim 23.
